(19) United States
(12) Patent Application Publication
(10) Pub. No.: US 2010/0294501 A1
(43) Pub. Date: Nov. 25, 2010

(54) PROCESS FOR THE PREPARATION OF AN ORGANIC COMPOSITION COMPRISING AN N-NONYL ESTER

(76) Inventors: Peter Daute, Beverstedt (DE); Anja Vonderhagen, Dusseldorf (DE); Heinz Muller, Monheim (DE)

Correspondence Address: SMITH MOORE LEATHERWOOD LLP, P.O. BOX 21927, GREENSBORO, NC, 27420, US

(21) Appl. No.: 12/743,878

(22) PCT Filed: Nov. 20, 2008
(86) PCT No.: PCT/EP08/65933

§ 371 Date: Jul. 23, 2010

(30) Foreign Application Priority Data
Nov. 20, 2007 (DE) 102007055595.6
Feb. 14, 2008 (DE) 102008009369.6

(51) Int. Cl. E21B 37/00; A61K 8/92; C08K 5/101; C12N 9/00; C07C 69/00; E21B 7/00; E21B 43/00

(52) U.S. Cl. 166/311; 424/70.1; 524/315; 435/183; 560/129; 175/57; 166/369

(57) ABSTRACT

The present invention relates to a process for the preparation of an organic composition which contains a functional component chosen from the group consisting of a thermoplastic polymer, an enzyme, a setting agent, a paraffin, an oil, a colouring agent and a hair or skin care substance, comprising as process steps of providing an n-nonyl ester as an additive, which is obtainable by reaction of an n-nonyl alcohol component with a further component which is capable of reacting with the n-nonyl alcohol component to form an n-nonyl ester, and the functional component, and optionally at least one further additive; and mixing of the n-nonyl ester, the functional component. and optionally the at least one further additive.

The present invention relates to a process for the preparation of an organic composition which comprises a functional component chosen from the group consisting of a thermoplastic polymer, an enzyme, a setting agent, a paraffin, an oil, a coloring agent and a hair care or skin care substance, and an n-nonyl ester, a process for the production of a shaped article, a process for the production of a packed product, the use of at least one n-nonyl ester, the use of a shaped article, a process for cleaning the surfaces of bore holes, drilling equipment or drill cuttings, processes for the production of a bore hole and processes for the production of an oil or a gas.

Linear fatty alcohols of short to medium chain length are successfully used nowadays as raw materials for surfactants, agents for influencing foam, solvents, agents for imparting consistency, lubricant additives and as etherification or esterification components in plastics processing. Either linear C₈- or C₁₀-alcohols or branched C₉-alcohols (i-nonanol) are available. The linear alcohols are usually of native origin and always even-numbered. C₈/C₁₀ fractions with 40 to 48 wt. % of C₈-alcohols and 51 to 59 wt. % of C₉-alcohols are preferably employed here.

Pure C₁₀-alcohol and derivatives thereof, such as, for example, ethers or esters, indeed have a high boiling point and therefore have a comparatively low volatility, but have high solidification points. Pure C₈-alcohol and derivatives thereof in turn are indeed characterized by low solidification points, but have low boiling points and are therefore very volatile.

The branched i-nonanols are mixtures of substances and are produced petrochemically. Branching of the alcohols leads to a poorer biodegradability. Disadvantages in connection with the use of i-nonanols are furthermore the too high a melting point or too low a melting range of the derivatives such as esters, ethoxylates and sulphates, and indeed also when alcohol mixtures are employed. Limits are therefore imposed on this product group due to the non-ideal viscosity properties, especially at lower temperatures.

The present invention was based on the object of at least partly overcoming the disadvantages emerging from the prior art.

In particular, the present invention was based on the object of providing a process with the aid of which organic compositions comprising esters of linear fatty alcohols of short to medium chain length as an additive can be provided, these organic compositions having less highly volatile components compared with the comparable organic compositions known from the prior art and also having satisfactory viscosity properties at low temperatures.

The present invention was moreover based on the object of providing a process with the aid of which organic compositions comprising esters of linear fatty alcohols of short to medium chain length as an additive can be provided, as many components as possible of these organic compositions being based on renewable raw materials or on educts which can be obtained from renewable raw materials.

The organic compositions obtainable by this process are moreover to have improved use properties compared with the organic compositions known from the prior art.

In particular, the present invention was based on the object of providing a compound which can also be employed in particular as an additive in drilling fluids or cleaning compositions for drilling equipment.

A contribution towards achieving at least one of the abovementioned objects is made by the subject matter of the category-forming claims, the sub-claims dependent upon these representing further embodiments according to the invention.

The present invention therefore relates in particular to a process for the preparation of an organic composition which comprises a functional component chosen from the group consisting of a thermoplastic polymer, an enzyme, a setting agent, a paraffin, an oil, a coloring agent and a hair or skin care substance, comprising as process steps:
i) provision
   ia) of an n-nonyl ester as an additive, which is obtainable by reaction of an n-nonyl alcohol component with a further component which is capable of reacting with the n-nonyl alcohol component to form an n-nonyl ester,
   ib) the functional component, and optionally
   ic) at least one further additive;
ii) mixing of the n-nonyl ester, the functional component and optionally the at least one further additive.

In the context of the present invention, a “functional component” is understood as meaning preferably a component which imparts to the composition to which this functional component is added its characteristic functional property. Thus, in the context of the present invention the functional component of a thermoplastic composition is the thermoplastic polymer, the functional component of an adhesive is the setting agent, the functional component of a lubricant formulation is the oil, the functional component of a detergent is the enzyme, the functional component of a defoamer is the paraffin, the functional component of a lacquer or a paint is the coloring agent and the functional component of a cosmetic formulation is the hair or skin care substance.

In the context of the present invention, an “organic composition” is understood as meaning preferably a composition which comprises organic components to the extent of more than 50 wt. %, based on the total weight of the organic composition, an organic component being understood as meaning preferably a carbon-containing compound with the exception of CO₂, CO, carbides, CSO and pure carbon compounds, such as graphite, carbon black or diamond. The organic component is preferably a hydrocarbon compound, which can contain oxygen, nitrogen, phosphorus, sulphur or at least two of these atoms as hetero atoms.

In step ia) of the process according to the invention, an n-nonyl ester, which is obtainable by reaction of an n-nonyl alcohol component with a further component which is capable of reacting with the n-nonyl alcohol component to form an n-nonyl ester, is provided as an additive.

This provision of an n-nonyl ester preferably comprises the following process steps:
ia1) provision of an n-nonyl alcohol component;
ia2) provision of a further component which is capable of reacting with the n-nonyl alcohol component to form an n-nonyl ester;
ia3) reaction of the n-nonyl alcohol component with the at least one further component to form an n-nonyl ester.

In process step 1a1) of the process for the provision of an n-nonyl ester, an n-nonyl alcohol component is first provided. According to a preferred embodiment of the process according to the invention for the preparation of an organic composition, it is preferable for the n-nonyl alcohol component to be obtained from pelargonic acid to the extent of at least 80 wt. %, particularly preferably to the extent of at least 90 wt. % and most preferably to the extent of at least 99 wt. %, in each case based on the n-nonyl alcohol component provided. In this connection it is furthermore preferable for the provision of the n-nonyl alcohol component to include the catalytic hydrogenation of pelargonic acid (octanecarboxylic acid, nonanoic acid), for example by the process described in WO-A-2006/021328, or the catalytic hydrogenation of the oleic acid ozonide obtained in the ozonolysis of oleic acid. The catalytic hydrogenation of esters of pelargonic acid, for example the catalytic hydrogenation of the methyl, ethyl, propyl or butyl ester of pelargonic acid, is furthermore conceivable. If the n-nonyl alcohol component is obtained by the catalytic hydrogenation of pelargonic acid, the pelargonic acid itself can be obtained, for example, by ozonolysis of oleic acid and subsequent oxidative working up of the oleic acid ozonide or by ozonolysis of erucic acid and subsequent oxidative working up of the erucic acid ozonide. Such a process is carried out on a large industrial scale, for example, by Unilever, Emery and Henkel and is also described, inter alia, in “Ozonierung von Alkenen in Alkoholen als Losungsmittel”, dissertation by Eberhard Rischbieter, University of Carolo-Wilhelmina in Braunschweig, 2000 or in U.S. Pat. No. 2,813,113. The oxidation of the aldehydes obtained in the oxidative working up of ozonides and the formation of the corresponding acid derivatives are described, for example, in DE-C-100 70 770. The preparation of oleic acid can in turn be carried out from tallow or tall oils, such as is described, for example, in U.S. Pat. No. 6,498,261. In addition to ozonolysis of oleic acid or of erucic acid, pelargonic acid can also be obtained by isomerization of petrochemical raw materials. Petrochemical production of pelargonic acid as described, for example, by Harold A., Wittcoff, Bryan G., Reuben, Jeffrey S. Plotkin in “Fats and Oils”, Industrial Organic Chemicals (Second Edition) (2004), John Wiley & Sons, Inc., pages 411-433, or the preparation of pelargonic acid from oleic acid by the process described in GB-A-813842 are furthermore conceivable.

According to a particular embodiment of the process according to the invention for the preparation of an organic composition, the n-nonyl alcohol component employed for the preparation of the n-nonyl ester comprises, in addition to the n-nonyl alcohol, further alcohols, for example C₈- and/or C₁₀-alcohols, it being particularly preferable in this case, however, for the n-nonyl alcohol component to contain less than 10 wt. %, particularly preferably less than 7.5 wt. % and most preferably less than 5 wt. %, in each case based on the n-nonyl alcohol component, of C₈- and C₁₀-alcohols. In the case where a mixture of n-nonyl alcohol and at least one further alcohol is employed, the content of n-nonyl alcohol in the n-nonyl alcohol component is preferably at least 90 wt. %, particularly preferably at least 92.5 wt. % and most preferably at least 95 wt. %, in each case based on the total weight of the n-nonyl alcohol component.

An n-nonyl alcohol component which is particularly preferred according to the invention is, in particular, that n-nonyl alcohol component which is obtained by catalytic hydrogenation of the pelargonic acid marketed under the brand names EMERY® 1202, EMERY® 1203 and EMERY® 1210, EMERY® 1202 comprising C₆-monocarboxylic acids to the extent of less than 1 wt. %, C₇-monocarboxylic acids to the extent of about 1 wt. %, C₈-monocarboxylic acids to the extent of about 4 wt. %, pelargonic acid to the extent of about 93 wt. % and other by-products, in particular monocarboxylic acids having more than 9 carbon atoms, to the extent of about 2 wt. %, EMERY® 1203 comprising C₆-C₈-monocarboxylic acids to the extent of about 0.1 wt. %, pelargonic acid to the extent of about 99 wt. % and other by-products, in particular monocarboxylic acids having more than 9 carbon atoms, to the extent of about 0.9 wt. %, and EMERY®1210 comprising C₅-monocarboxylic acids to the extent of about 3 wt. %, C₆-monocarboxylic acids to the extent of about 27 wt. %, C₇-monocarboxylic acids to the extent of about 31 wt. %, C₈-monocarboxylic acids to the extent of about 12 wt. % and pelargonic acid to the extent of about 27 wt. %, the use of EMERY® 1203 being particularly preferred, however, since the content of pelargonic acid is particularly high here. n-Nonyl alcohol components which have been obtained by catalytic hydrogenation of pelargonic acid mixtures which contain more than 10 wt. %, particularly preferably more than 25 wt. % of pelargonic acid are furthermore advantageous in principle.

In process step ia2) of the process for the provision of an n-nonyl ester, at least one further component which is capable of reacting with the n-nonyl alcohol component to form an n-nonyl ester is provided, this further component preferably being an inorganic acid, in particular an inorganic acid chosen from the group consisting of sulphuric acid, sulphurous acid, phosphoric acid or phosphorous acid, or an organic acid, in particular an organic acid chosen from the group consisting of monocarboxylic acids, dicarboxylic acids, tricarboxylic acids, tetracarboxylic acids or derivatives of the abovementioned carboxylic acids.

The term “carboxylic acid” as used herein includes the carboxylic acid in its protonated form, the carboxylic acid in its deprotonated form (that is to say, in particular, salts of the carboxylic acid) and also mixtures of the carboxylic acid in its protonated form and its deprotonated form. The term “carboxylic acid” furthermore in principle includes all compounds which comprise at least one carboxylic acid group. It therefore also includes in particular compounds which, in addition to the at least one carboxylic acid group, also comprise other functional groups, such as, for example, hydroxyl groups, keto groups or ether groups.

The term “derivative of a carboxylic acid” includes all derivatives of a carboxylic acid which lead to a corresponding ester of the carboxylic acid in a reaction with an alcohol. In particular, the term “derivative of a carboxylic acid” includes the acid chlorides of the carboxylic acid and the acid anhydrides of the carboxylic acid. These derivatives preferably have an increased reactivity of the carboxylic acid group compared with the carboxylic acid, so that during a reaction with an alcohol the ester formation is promoted.

The use of mono-, di-, tri-, tetra- or polycarboxylic acids having more than four carboxyl groups, a derivative of such a carboxylic acid or a mixture of such a carboxylic acid and a derivative of such a carboxylic acid as a further component is particularly preferred according to the invention. In this context, possible carboxylic acids are, in particular, saturated or unsaturated carboxylic acids having a number of carbon atoms in a range of from 6 to 26, particularly preferably in a range of from 8 to 24, still more preferably in a range of from 10 to 22, even more preferably in a range of from 12 to 20 and most preferably in a range of from 14 to 18. Fatty acids are therefore carboxylic acids which are particularly preferred according to the invention.

Examples of suitable carboxylic acids include, in particular, caproic acid, oenanthic acid, caprylic acid, pelargonic acid, capric acid, lauric acid, myristic acid, fish oil, palmitic acid, palmoleic acid, pelagonic acid, margaric acid, stearic acid, elaeostearic acid, isostearic acid, isotridecanoic acid, arachic acid, behenic acid, lignoceric acid, cerotic acid, undecylenic acid, oleic acid, elaidic acid, vaccenic acid, icosenic acid, rapeseed oil, cetoleic acid, erucic acid, nervonic acid, linoleic acid, linolenic acid, arachidonic acid, timnodonic acid, clupanodonic acid, petroselic acid, gadoleic acid or cervonic acid.

In addition to the abovementioned fatty acids, di-, tri- or tetracarboxylic acids or anhydrides thereof chosen from the group consisting of phthalic anhydride, isophthalic acid, phthalic acid, terephthalic acid, tetrahydrophthalic anhydride, hexahydrophthalic anhydride, naphthalenedicarboxylic acid, 4,4′-biphenyldicarboxylic acid, diphenylmethane-4,4′-dicarboxylic acid, succinic acid, fumaric acid, adipic acid, sebacic acid, azelaic acid, trimellitic acid, pyromellitic acid, mellitic acid and maleic anhydride are also possible, of these acids adipic acid, trimellitic acid, terephthalic acid and azelaic acid being particularly preferred.

Hydroxycarboxylic acids are furthermore also included as suitable carboxylic acids, and among these, hydroxy-fatty acids, such as, for example, ricinoleic acid, 12-hydroxystearic acid, hydrogenated castor oil fatty acids (fatty acids which contain small amounts of stearic acid and palmitic acid, just as 12-hydroxystearic acid), sabinic acid, 2-hydroxytetradecanoic acid, ipurolic acid (3,11-dihydroxytetradecanoic acid), 2-hydroxyhexadecanoic acid, jalapinolic acid, juniperic acid, ambrettolic acid, aleuritic acid, 2-hydroxyoctadecanoic acid, 18-hydroxyoctadecanoic acid, 9,10-dihydroxyoctadecanoic acid, kamiolenic acid, ferronic acid, cerebronic acid, 9-hydroxystearic acid and 10-hydroxystearic acid, are particularly preferred and 12-hydroxystearic acid and ricinoleic acid are most preferred. Short-chain hydroxycarboxylic acids, such as, for example, lactic acid, 3-hydroxypropionic acid, 2-hydroxybenzoic acid, 3-hydroxybenzoic acid or 4-hydroxybenzoic acid, are furthermore also suitable.

The abovementioned fatty acids can be obtained from naturally occurring fats and oils, for example via fat cleavage at elevated temperature under increased pressure and subsequent separation of the fatty acid mixtures obtained, optionally after hydrogenation of the double bonds present. Technical grade fatty acids, which as a rule are mixtures of various fatty acids of a certain chain length range with one fatty acid as the main constituent, are preferably employed here. Fatty acids having 12 to 18 C atoms, by themselves or in a mixture, are preferably provided.

In process step ia3) of the process for the provision of an n-nonyl ester, the n-nonyl alcohol component is reacted with the at least one further component to form an n-nonyl ester.

The preparation of the n-nonyl ester can be carried out by any process for the preparation of such an ester known to the person skilled in the art.

Preferably, for preparation of the esters, the n-nonyl alcohol component and the carboxylic acid or the derivative of the carboxylic acid are initially introduced into the reaction vessel and the reaction is then catalyzed in the presence of a suitable esterification catalyst. Esterification catalysts which can be employed are acids, such as, for example, sulphuric acid or p-toluenesulphonic acid, or metals and compounds thereof. For example, tin, titanium or zirconium, which can be used as finely divided metals or expediently in the form of their salts, oxides or soluble organic compounds, are suitable. In contrast to proton acids, the metal catalysts are high temperature catalysts which as a rule achieve their full activity only at temperatures above 180° C. However, they are preferred according to the invention because they produce fewer by-products, such as, for example, olefins, compared with proton catalysis. Esterification catalysts which are particularly preferred according to the invention are one or more divalent tin compounds, or tin compounds or elemental tin which can react with the educts to give divalent tin compounds. For example, tin, tin(II) chloride, tin(II) sulphate, tin(II) alcoholates or tin(II) salts of organic acids, in particular of mono- and dicarboxylic acids, can be employed as the catalyst. Particularly preferred tin catalysts are tin(II) oxalate and tin(II) benzoate.

The esterification reaction between the n-nonyl alcohol component and the carboxylic acid or the derivative of the carboxylic acid can be carried out by processes known to the person skilled in the art. In this context, it may be advantageous in particular to remove the water formed in the reaction from the reaction mixture, this removal of the water preferably being carried out by distillation, optionally by distillation with alcohol employed in excess. Alcohol which has not reacted after the esterification reaction has been carried out can also be removed from the reaction mixture, this removal of the alcohol preferably also be carried out by means of distillation. When the esterification reaction has ended, in particular after the unreacted alcohol has been separated off, the catalyst remaining in the reaction mixture can furthermore be separated off by a filtration or by centrifugation, optionally after treatment with a base.

It is furthermore preferable for the esterification reaction between the n-nonyl alcohol component and the carboxylic acid or the derivative of the carboxylic acid to be carried out at a temperature in a range of from 50 to 300° C., particularly preferably in a range of from 100 to 275° C. and most preferably in a range of from 200 to 250° C. The optimum temperatures depend on the alcohol(s) employed, the progress of the reaction, the catalyst type and the catalyst concentration. It can be easily determined by experiments for each individual case. Higher temperatures increase the rates of reaction and promote side reactions, such as, for example, splitting off of water from alcohols or the formation of colored by-products. The desired temperature or the desired temperature range can be established by the pressure in the reaction vessel (slightly increased pressure, normal pressure or optionally reduced pressure).

According to a particular embodiment of the process for the provision of an n-nonyl ester, this can comprise as a further process step the step of
Ia4) alkoxylation, preferably ethoxylation or propoxylation, of the n-nonyl ester to form an alkoxylated, preferably an ethoxylated or propoxylated n-nonyl ester having preferably 2 to 50, particularly preferably having 4 to 25 ether recurring units

wherein these ether recurring units are preferably a —[O—CH₂—CH₂]— unit, a —[O—CH₂—CH₂—CH₂]— unit or a mixture of these units.

Such alkoxylated n-nonyl esters can be obtained, for example, by reacting the n-nonyl ester with ethylene oxide, propylene oxide or a mixture of ethylene oxide and propylene oxide in the presence of suitable catalysts in relative amounts such that 2 to 50 ether recurring units, particularly preferably 4 to 25 ether recurring units are inserted into the ester bond. A process with which esters can be alkoxylated is described, for example, in DE-A-40 10 606, the disclosure content of which with respect to the alkoxylation of esters is introduced herewith as reference and represents part of the disclosure of the present invention.

In process step ib) of the process according to the invention for the preparation of an organic composition, a functional component is provided.
1. According to a first variant of the process according to the invention, the functional component is a thermoplastic polymer and the organic composition is consequently a thermoplastic organic composition.
   The term “thermoplastic polymer” as used herein is understood as meaning plastics which can be simply (thermoplastically) deformed in a certain temperature range. This operation is reversible and can be repeated by cooling and reheating to the molten state as often as desired, as long as thermal decomposition of the material does not start due to overheating.
   Possible thermoplastic polymers which can be employed as the functional component according to the first variant of the process according to the invention are generally polycondensates or chain polymers or a mixture of these two, in particular thermoplastic polyurethanes, thermoplastic polyesters, thermoplastic polyamides, thermoplastic polyolefins, thermoplastic polyvinyl esters, thermoplastic polyethers, thermoplastic polystyrenes, thermoplastic polyimides, thermoplastic sulphur polymers, thermoplastic polyacetals, thermoplastic fluorinated plastics, thermoplastic styrene/olefin copolymers, thermoplastic polyacrylates, thermoplastic ethylene/vinyl acetate copolymers or mixtures of two or more of the abovementioned thermoplastic polymers.
   However, it is preferable according to the invention for the thermoplastic polymer to be based on thermoplastic polyesters to the extent of more than 90 wt. %, particularly preferably to the extent of more than 95 wt. %, even still more preferably to the extent of at least 99 wt. % and most preferably to the extent of 100 wt. %, in each case based on the total weight of the thermoplastic polymer. The term “polyester” as used herein includes in particular polymers which have been obtained by a polycondensation reaction between a polycarboxylic acid and a polyol (so-called “AA//BB polyesters”) or by a polycondensation reaction of a hydroxycarboxylic acid or by ring-opening polymerization of a cyclic ether (so-called “AB polyesters”). In one embodiment according to the invention, polycarbonates which are obtainable by reaction of phosgene with diols may be excluded from the term “polyester” used according to the invention.
   In principle, all the thermoplastic polyesters and copolyesters currently known can be used. Examples of such polyesters include essentially linear polyesters which have been prepared via a condensation reaction of at least one polycarboxylic acid, preferably a dicarboxylic acid (dibasic acid) or an ester-forming derivative thereof and at least one polyol, preferably a dihydric alcohol (diol). The preferably dibasic acid and the preferably dihydric diol can both be either aliphatic or aromatic, aromatic and partly aromatic polyesters being preferred as thermoplastic molding materials, however, in view of their high softening points and stability to hydrolysis. In the case of aromatic polyesters, substantially all the ester linkages are added on to the aromatic rings. They can be semicrystalline and even show liquid crystal properties or be amorphous. Partly aromatic polyesters which have been obtained from at least one aromatic dicarboxylic acid or an ester-forming derivative thereof and at least one aliphatic diol are thermoplastic polyesters which are particularly preferred according to the invention. Examples of suitable aromatic dicarboxylic acids include terephthalic acid, 1,4-naphthalenedicarboxylic acid or 4,4′-biphenyldicarboxylic acid. Examples of suitable aliphatic diols include alkylene diols, specifically those which contain 2 to 6 C atoms, preferably 2 to 4 C atoms, where ethylene glycol, propylene diols and butylene diols are to be mentioned in particular here. Ethylene glycol, 1,3-propylene diol or 1,4-butylene diol are preferably used as the polyol or diol component for the preparation of the thermoplastic polyesters comprised as component a) in the composition according to the invention. Thermoplastic polyesters which are obtainable by reaction of a dicarboxylic acid with a diol and are particularly preferred according to the invention include, in particular, polyalkylene terephthalates, for example polyethylene terephthalate (PET), polypropylene terephthalate (PPT) or polybutylene terephthalate (PBT), polyalkylene naphthalates, for example polyethylene naphthalate (PEN) or polybutylene naphthalate (PBN), polylactic acid (PLA), polyalkylene dibenzoates, for example polyethylene dibenzoate, and mixtures of at least two of these thermoplastic polyesters.
   These partly aromatic polyesters described above can optionally comprise a small amount of units which originate from other dicarboxylic acids, for example isophthalic acid, or other diols, such as cyclohexanedimethanol, which in general reduces the melting point of the polyester. A specific group of partly aromatic polyesters are so-called segmented or block copolyesters, which in addition to the above-mentioned polyester segments (also called “hard segments”), contain so-called “soft segments”. These soft segments originate from a flexible polymer, that is to say an essentially amorphous polymer with a low glass transition temperature (T_{g}) and low rigidity, having reactive end groups, preferably two hydroxyl groups. Preferably, the glass transition temperature of these “soft segments” is below 0° C., particularly preferably below −20° C. and most preferably below −40° C. In principle, several different polymers can be used as the soft segment. Suitable examples of “soft segments” are aliphatic polyethers, aliphatic polyesters or aliphatic polycarbonates. The molecular weight of the soft segments can vary within wide limits, but is preferably between 400 and 6,000 g/mol.
   In addition to the abovementioned linear polyesters which are obtainable via a polycondensation reaction of at least one polycarboxylic acid or an ester-forming derivative thereof and at least one polyol, thermoplastic polyesters which are obtainable by polycondensation reaction of short-chain hydroxycarboxylic acids or by ring-opening reaction of cyclic esters can also be employed as the main component according to the first variant of the process according to the invention.
   Examples of suitable short-chain hydroxycarboxylic acids which can be employed for the preparation of thermoplastic polymers include in particular L-lactic acid, D-lactic acid, DL-lactic acid, glycolic acid, 3-hydroxybutyric acid, 4-hydroxybutyric acid, 4-hydroxyvaleric acid, 5-hydroxyvaleric acid, 6-hydroxycaproic acid and mixtures of these hydroxycarboxylic acids. Examples of suitable cyclic esters include in particular glycolide (a dimer of glycolic acid) and ε-caprolactone (a cyclic ester of 6-hydroxycaproic acid).
   The preparation of the thermoplastic polyesters described above is also described, inter alia, in “Encyclopedia of Polymer Science and Engineering”, volume 12, pages 1 to 75 and pages 217 to 256; John Wiley & Sons (1988) and also in “Ullmann's Encyclopedia of Industrial Chemistry”, volume A21, pages 227 to 251, VCH Publishers Inc. (1992). Thermoplastic polymers which are preferred according to the invention are polyethylene terephthalate (PET), polybutylene terephthalate (PBT) and polylactic acid (PLA). According to the first variant of the process according to the invention, it is furthermore preferable that this thermoplastic polymer can be employed as the functional component in an amount of at least 60 wt. %, preferably of at least 75 wt. % and particularly preferably of at least 90 wt. %, in each case based on the total weight of the organic composition, while the n-nonyl ester is employed as an additive, in particular as a mould release agent, as an antifogging agent, as a plasticizer, as an antistatic or as a lubricant, most preferably in a function as a mould release agent, preferably in an amount in a range of from 0.001 to 40 wt. %, particularly preferably in an amount in a range of from 0.01 to 25 wt. % and most preferably in an amount in a range of from 0.1 to 10 wt. %, in each case based on the total weight of the thermoplastic composition.
   Possible additives which, according to this first variant of the process according to the invention, can be provided in process step ic) are, in particular, impact modifiers, filler materials, reinforcing agents, flame retardant compounds, heat and UV stabilizers, antioxidants, other processing auxiliary substances, nucleating agents, dyestuffs and antidripping agents. Examples of suitable impact modifiers, filler materials, reinforcing agents and flame retardant compounds are to be found, inter alia, in US 2005/0234171 A1. These further additives are preferably employed in an amount in a range of from 0.001 to 20 wt. %, particularly preferably in an amount in a range of from 0.01 to 10 wt. % and most preferably in an amount in a range of from 0.1 to 5 wt. %, in each case based on the total weight of the thermoplastic composition.
   The mixing of the n-nonyl ester, the functional component (thermoplastic polymer) and optionally the additive carried out process step ii) in the case of the first variant of the process according to the invention can be carried out using known techniques. Thus, the mixing can be, for example, a dry mixing operation, in which the various components are mixed at below the melt processing temperature of the thermoplastic polymer, or a melt mixing process, in which the components are optionally premixed, and mixed at the melt processing temperatures of the thermoplastic polymer. The melt mixing processes include, in particular, the melt kneading process, which is preferred according to the invention and can be realized, for example, by continuous melt kneading using a single-screw kneading machine, a twin-screw kneading machine of the toothed-same direction of rotation type, toothed-opposite direction of rotation type, nontoothed-same direction of rotation type, nontoothed-opposite direction of rotation type, or other types, or by batch melt kneading using a roller kneading machine, a Banbury kneading machine or similar. A combination of a dry mixing process and a melt mixing process is furthermore conceivable.
   The sequence and the nature and manner of the addition of the individual components ia), ib) and optionally ic) into the mixing device is furthermore in principle not critical. Thus, for example, the thermoplastic polymer and optionally the additives can first be initially introduced into the mixing device and the n-nonyl ester only then added. It is also conceivable for the n-nonyl ester or a part of the n-nonyl ester first to be mixed with one or more other components of the thermoplastic composition according to the invention, for example with one or more additives, and then either for this mixture to be added to the thermoplastic polymer already in the mixing device, or for this mixture first to be initially introduced into the mixing device and the thermoplastic polymer only then added.
   In further embodiments of the process according to the invention for the preparation of an organic composition according to the first variant of the process according to the invention, mixing is carried out in accordance with at least one of the following measures:
   M1) at the glass transition temperature of the thermoplastic polymer or at a temperature above the glass transition temperature of the thermoplastic polymer;
   M2) wherein the n-nonyl ester is more liquid than the thermoplastic polymer; or
   M3) wherein at least a part of the n-nonyl is added to the precursor of the thermoplastic polymer.
   Embodiments according to the invention furthermore include combining of two or more of the above measures. The following combinations of measures represented with the aid of combination of figures thus result specifically as embodiments: M1M2, M1M3, M2M3 and M1M2M3.
   According to a preferred embodiment M1 of the process according to the invention, the mixing of the components provided in process steps ia), ib) and optionally ic) is carried out in process step ii) of the process according to the invention by a melt mixing process. In this connection it is preferable in particular for the mixing in process step ii) to be carried out at the glass transition temperature of the thermoplastic polymer or at a temperature above the glass transition temperature of the thermoplastic polymer. In this connection, it is particularly preferable for the mixing to be carried out at a temperature in a range of from 5 degrees below the glass transition temperature (T_{g}) to 200° C. above the glass transition temperature of the thermoplastic polymer employed, particularly preferably at a temperature in a range of from 1 degree below the glass transition temperature (T_{g}) to 180° C. above the glass transition temperature of the thermoplastic polymer employed and most preferably at a temperature in a range of from 1 degree above the glass transition temperature (T_{g}) to 150° C. above the glass transition temperature of the thermoplastic polymer employed, the upper limit of the temperature range being essentially limited by the decomposition temperature of the thermoplastic polymer employed. Embodiments according to the invention furthermore include mixing at temperatures in a range of from 10 to 180° C. and preferably 50 to 150° C. above the glass transition temperature of the thermoplastic polymer employed.
   In embodiment M2 according to the invention, in which the n-nonyl ester is more liquid than the thermoplastic polymer, it is preferable for the n-nonyl ester to be employed at a temperature at which it is liquid and the thermoplastic polymer is not yet liquid. The temperature of the thermoplastic polymer here is preferably below the glass transition temperature of this polymer. It is thus preferable for the melting temperature of the n-nonyl ester and the glass transition temperature of the thermoplastic polymer to differ by at least 5° C., preferably at least 10° C. and particularly preferably at least 30° C. In this embodiment and also generally, it is furthermore preferable for the thermoplastic polymer to be employed as granules. In general, all granule forms known to the person skilled in the art with a spherical or cylindrical spatial shape are also possible in the present case. The granule size, determined by means of sieve analysis, is in a range of from 0.01 to 5 cm, and preferably in a range of from 0.1 to 4 cm for at least 70 wt. % of the granule particles. By the procedure according to this embodiment, the surfaces of the granule particles can be at least partly coated with the n-nonyl ester, so that at least partly coated thermoplastic polymer granules are obtained. This allows a distribution of the n-nonyl ester according to the invention in the thermoplastic composition which is as homogeneous as possible, especially if this is made up as a formulation for the extrusion taking place later.
   In embodiment M3 according to the invention, in which the n-nonyl ester is added to the precursor of the thermoplastic polymer, n-nonyl esters in the liquid form and also in the solid form are possible. Possible precursors of the thermoplastic polymer are in principle all the precursors before the thermoplastic polymer is obtained which are known to the person skilled in the art. These include, in particular, precursors which have a lower molecular weight than the final thermoplastic polymer. It is preferable here for the molecular weight of the precursor to differ from that of the finished thermoplastic polymer by a factor of at least 1.1, preferably at least 1.5 and particularly preferably at least by a factor of 2. In addition to the monomers and oligomers, which preferably comprise 2 to 100 monomers, employed for the preparation of the thermoplastic polymer, a prepolymer which is polymerized completely, usually by heat treatment, to give the finished thermoplastic polymer is included, especially in the case of polycondensates. The prepolymer is preferably based on more than 100 monomers as recurring units, the number of the monomers as recurring units and therefore the final molecular weight of the finished thermoplastic polymer not being reached. It is therefore particularly preferable for the n-nonyl ester in each case to be added to the monomers, oligomers or the prepolymer or at least two of these. By this means, in addition to a homogeneous distribution of the n-nonyl ester, incorporation of the n-nonyl ester by chemical bonds with the thermoplastic polymer is also achieved, usually by the conditions prevailing during the polymerization or complete polymerization.
2. According to a second variant of the process according to the invention, the functional component is an enzyme and the organic composition is a detergent.
   Suitable enzymes are, in particular, amylases, proteases, lipases, cellulases, peroxidases or mixtures of at least two of these enzymes.
   Amylases are added to remove starch and glycogen. alpha-, beta- and gamma-amylases as well as glucoamylases and maltogenic amylases can be used according to the invention. Suitable amylases are commercially obtainable, for example under the names Duramyl®, Termamyl®, Fungamyl® and BAN® (Novo Nordisk), and Maxamyl®, or Purafect® OxAm. The amylases can originate from any desired sources, such as, for example, from bacteria, fungi, pancreatic glands of animal origin, from germinated cereal or from yeasts. Genetically modified amylases can also be used, optionally even preferably, as the functional component in the organic compositions according to the invention. The amylase enzymes can be present in the compositions according to the invention in an amount of from 0.0001 wt. % to 5 wt. %, particularly preferably from 0.0001 wt. % to 1 wt. % and most preferably from 0.0005 to 0.5 wt. %, in each case based on the total weight of the organic composition.
   In addition to amylases, according to the second variant of the process according to the invention, proteases for cleavage of proteins and peptide residues can also be added to the organic compositions according to the invention. Proteases are suitable in particular for hydrolytic cleavage and removal of protein residues, in particular dried-on protein residues. Proteases which are suitable according to the invention are proteinases (endopeptidases) and peptidases (exopeptidases). Proteases which can be used can be of plant, animal, bacterial and/or fungal origin. Suitable proteases are, in particular, serine, cysteine, aspartate and metal proteases. Genetically modified proteases can also be used, optionally even preferably, in the organic compositions according to the invention. Proteases which can be used are obtainable commercially under the names Alcalase®, BLAP®, Durazym®, Esperase®, Everlase®, Maxapem®, Maxatase®, Optimase Purafect® OxP or Savinase®. Proteases are conventionally employed in an amount of from 0.00001 to 1.5 wt. % and particularly preferably from 0.0001 to 0.75 wt. %, in each case based on the total weight of the organic composition.
   Lipases can also be employed as the functional component according to the second variant of the process according to the invention. They serve to remove firmly adhering fat grime. Lipases are thus a bioalternative to surfactants and, in the range of from 0.0001 to 1 wt. %, based on the total weight of the organic composition, can assist the cleaning action of surfactants. Suitable lipases can be obtained from plants (for example Rhizinus species), microorganisms and animal sources, such as, for example, pancreatic lipases. Commercially obtainable lipases are, for example, Lipolase®, Lipomax®, Lipozym® and Lumafast®.
   The abovementioned enzymes can optionally be combined with any desired other enzymes in order to improve further the cleaning power of the organic composition employed as a detergent. Further enzymes which are suitable according to the invention are cellulases, hemicullulases, peroxidases, reductases, oxidases, ligninases, cutinases, pectinases, xylanases, phenoloxidases, lipoxygenases, tannases, pentosanases, malanases, glucanases, arabinosidases and any desired mixtures of these enzymes.
   In this second variant of the process according to the invention, the n-nonyl ester is preferably added to the detergent in the function of a surfactant, it being preferable in this case for the n-nonyl ester to be employed in an amount of from 0.001 to 40 wt. %, particularly preferably from 0.01 to 30 wt. %, still more preferably from 0.1 to 20 wt. % and most preferably from 1 to 10 wt. %, in each case based on the total weight of the organic composition.
   Possible additives which can be provided, according to this second variant of the process according to the invention, in process step ic) are, in particular, further surfactants which differ from the n-nonyl ester, builders, solvents, hydrophobic components, phase separation auxiliary substances, thickening agents, polymers, soil release active compounds, solubilizing agents, hydrotropic agents, such as, for example, sodium cumenesulphonate, octyl sulphate, butyl glucoside and butyl glycol, emulsifiers, such as, for example, gall soap, gloss-drying additives, cleaning intensifying agents, antimicrobial active compounds or disinfectants, antistatics, preservatives, such as, for example, glutaraldehyde, bleaching systems, perfumes, fragrances, dyestuffs, opacifying agents or also skin protection agents, the amount of such additives conventionally not being above 12 wt. %, based on the total weight of the organic composition.
   An overview of the additives contained in detergents, of the amounts in which these are added to the detergents and of the nature and manner of the preparation of a detergent from the abovementioned components in the context of process step ii) of the process according to the invention for the preparation of an organic composition can be found, inter alia, in DE 101 06 712 A1.
3. According to a third variant of the process according to the invention, the functional component is a setting agent and the organic composition is an adhesive.
   The chemical composition of the setting agent contained in the adhesive depends on the manner in which setting by the adhesive takes place. It can thus be a physically setting adhesive, for example a hot-melt adhesive containing, for example, ethylene/vinyl acetate copolymers, polyamides or polyesters as setting agents, a solvent-containing wet adhesive containing, for example, polymeric vinyl compounds, polymethyl methacrylate or natural and synthetic rubber as the setting agent, a contact adhesive containing, for example, polychloroprenes or butadiene/acrylonitrile rubber as the setting agent, a dispersion adhesive containing, for example, polyvinyl acetate, vinyl acetate copolymers, polyacrylates, polyvinylidene chloride, styrene/butadiene copolymers, polyurethanes, polychloroprene or rubber latices as the setting agent, a water-based adhesive containing, for example, glutin sizes, such as, for example, hide size or fish size, sizes based on plant natural products, such as, for example, starch size, methylcellulose or casein size, or PVAL adhesives as the setting agent, a pressure-sensitive adhesive containing, for example, polyacrylates, polyvinyl ethers or natural rubber as the setting agent, or a plastisol containing, for example, PVC and plasticizer as the setting agent.
   The adhesive can furthermore be a chemically curing adhesive, for example a cyanoacrylate-based adhesive containing, for example, cyanoacrylic acid esters as the setting agent, a methyl methacrylate-based adhesive containing, for example, methacrylic acid methyl ester as the setting agent, an anaerobically curing adhesive containing, for example, diacrylic acid esters of diols as the setting agent, a radiation-curing adhesive containing, for example, epoxyacrylates or polyester acrylates as the setting agent, a phenol/formaldehyde resin-based adhesive containing, for example, phenols and formaldehyde as the setting agent, a silicone-based adhesive containing, for example, polyorganosiloxanes as the setting agent, a polyimide-based adhesive containing, for example, aromatic tetracarboxylic acid anhydrides and aromatic diamines as the setting agent, an epoxy resin adhesive containing, for example, oligomeric diepoxides and polyamines or polyamidoamines as the setting agent, or a polyurethane-based adhesive containing, for example, di- and optionally trifunctional isocyanates and polyols as the setting agent.
   The concentration of setting agent in the adhesive depends on the nature of the adhesive employed but is conventionally in a range of from 10 to 100 wt. %, particularly preferably from 20 to 90 wt. % and even more preferably from 30 to 80 wt. %, in each case based on the total weight of the adhesive.
   In this third variant of the process according to the invention, the n-nonyl ester is preferably added to the adhesive in the function of a solvent, an agent which imparts consistency or in the function of a surfactant, it being preferable in this case for the n-nonyl ester to be employed in an amount of from 0.001 to 40 wt. %, particularly preferably from 0.1 to 30 wt. %, still more preferably from 1 to 20 wt. % and most preferably from 3 to 10 wt. %, in each case based on the total weight of the organic composition. In particular, if the n-nonyl ester is employed as a solvent, for example in solvent-containing wet adhesives, the concentration of the n-nonyl ester can optionally also be above the abovementioned concentration ranges.
   The additives which, according to this third variant of the process according to the invention, can be provided in process step ic) depend on the nature of the particular adhesive. Possible additives are, in particular, fillers, such as, for example, chalk, natural ground or precipitated calcium carbonates, calcium magnesium carbonates (dolomite), silicates, such as, for example, aluminum silicates, barite or magnesium aluminum silicates and talc, and reinforcing fillers, such as, for example, carbon blacks, in particular flame blacks, channel blacks, gas blacks, furnace blacks or mixtures thereof, plasticizers or plasticizer mixtures, catalysts (in the case of chemically setting adhesives), stabilizers and solvents. The amount of such additives depends on the nature of the particular adhesive and is conventionally not more than 50 wt. %, based on the total weight of the organic composition.
   An overview of adhesives is given, inter alia, in the publication “Kleben/Klebstoffe” of the Fonds der Chemischen Industrie in the Verband der chemischen Industrie e.V., 2001.
4. According to a fourth variant of the process according to the invention, the functional component is a paraffin, in particular a paraffin wax, and the organic composition is a defoamer.
   The paraffin provided as the functional component in process step ib) in the fourth variant of the process according to the invention is in general a complex substance mixture without a sharp melting point. Characterization is conventionally by determination of its melting range by differential thermal analysis (DTA), as described in “The Analyst” (1962), 420, and/or its solidification point. This is understood as meaning the temperature at which the waxes passes from the liquid into the solid state by slow cooling. Paraffins having less than 17 C atoms cannot be used according to the invention, and their content in the paraffin wax mixture should therefore be as low as possible and is preferably below the limit which can be measured significantly by conventional analytical methods, for example gas chromatography. Waxes which solidify in the range of from 20° C. to 70° C. are preferably used. In this context, it is to be noted that paraffin wax mixtures which appear to be solid at room temperature can also contain various contents of liquid paraffin. In the paraffin waxes which can be used according to the invention, the liquid content at 40° C. is as high as possible, without already being 100% at this temperature. Particularly preferred paraffin wax mixtures have a liquid content at 40° C. of at least 50 wt. %, in particular from 55 wt. % to 80 wt. %, and a liquid content at 60° C. of at least 90 wt. %. As a consequence of this, the paraffins are flowable and pumpable at temperatures down to at least 70° C., preferably down to at least 60° C. It is moreover to be ensured that the paraffins as far as possible contain no volatile contents. Preferred paraffin waxes contain less than 1 wt. %, in particular less than 0.5 wt. % of contents which can be evaporated at 110° C. under normal pressure. Paraffin waxes which can be used according to the invention can be obtained, for example, under the trade names Lunaflex® from Fuller and Deawax® from DEA Mineralöl AG. The amount of paraffin in the organic composition acting as a defoamer is preferably in a range of from 50 to 99 wt. %, particularly preferably from 60 to 95 wt. % and most preferably from 70 to 95 wt. %, in each case based on the total weight of the organic composition. However, if carrier materials are added to the defoamer, the content of paraffin can also be significantly below the abovementioned concentration ranges.
   In this fourth variant of the process according to the invention, the n-nonyl ester is preferably added to the defoamer in the function of a solvent or in the function of a surfactant, it being preferable in this case for the n-nonyl ester to be employed in an amount of from 0.001 to 20 wt. %, particularly preferably from 0.1 to 10 wt. %, still more preferably from 1 to 8 wt. % and most preferably from 2 to 7 wt. %, in each case based on the total weight of the organic composition.
   The additives which, according to this fourth variant of the process according to the invention, can be provided in process step ic) can be, for example, silicone oils and mixtures thereof with hydrophobized silica, or further compounds having a defoaming action, such as, for example, bisamides. The defoamer can also contain carrier materials, which preferably have a granular structure and comprise water-soluble or water-dispersible, surfactant-free compounds, in particular inorganic and/or organic salts which are suitable for use in detergents and cleaning compositions. Examples of water-soluble inorganic carrier materials which may be mentioned are, in particular, alkali metal carbonate, alkali metal borate, alkali metal alumosilicate and/or alkali metal sulphate, while organic carrier materials which can be employed are, for example, acetates, tartrates, succinates, citrates, carboxymethylsuccinates and the alkali metal salts of aminopolycarboxylic acids, such as EDTA, hydroxyalkane phosphonates and aminoalkane polyphosphonates, such as 1-hydroxyethane 1,1-diphosphonate, ethylenediaminotetramethylene phosphonate and diethylenetriaminepentamethylene phosphonate. The use of film-forming polymers, such as, for example, polyethylene glycols, polyvinyl alcohols, polyvinylpyrrolidones, polyacrylates and cellulose derivatives, as carrier materials is also conceivable. The amount of such additives is conventionally not more than 25 wt. %, based on the total weight of the organic composition. However, carrier materials can also be employed in a significantly higher concentration.
   The defoamers mentioned in WO-A-1997/034983 may be mentioned as an example of a defoamer which can be prepared according to the fourth variant of the process according to the invention, in particular the disclosure content of this publication with respect to the process for the preparation of a defoamer from the component provided in process steps 1a), 1b) and 1c) in the context of process step ii) also being introduced herewith as reference and forming part of the disclosure of the present invention.
5. According to a fifth variant of the process according to the invention, the functional component is an oil, preferably a hydrocarbon having 20 to 35 carbon atoms (lubricating oil), and the organic composition is a lubricant formulation.
   The oil contained in the lubricant formulation can be a refined product which has been obtained by separating off the hydrocarbon atoms having 20 to 35 carbon atoms naturally present in crude oil, a hydrocracking oil (HC synthesis oil) which has been obtained by cracking crude oil constituents which contain more than 35 carbon atoms, or synthetic hydrocarbons, which are obtained by cracking crude oil constituents having less than 12 carbon atoms to give gases such as, in particular, ethene or butene and subsequent synthesis of hydrocarbons having 20 to 35 carbon atoms from these gases.
   In addition to these oils, the lubricant formulation can also comprise bio-oils obtained from renewable raw materials, it being possible to employ in particular bio-oils from the HETG, HEPG, HEPR or the HEES group (VDMA 24568 ISO standard 15380). The HETG group includes triglycerides, such as, for example, rapeseed oil, while the HEPG group includes polyglycols. Synthetic esters, in particular TMP esters (trimethylpropane esters, also called oleic acid esters or trioleate) belong to the HEES group. The HEPR group includes liquids which mostly comprise polyalphaolefines (PAO) and related hydrocarbons.
   The amount of oil in the lubricant formulation is preferably in a range of from 50 to 99 wt. %, particularly preferably in a range of from 60 to 95 wt. % and most preferably in a range of from 70 to 90 wt. %, in each case based on the total weight of the organic composition. However, if the lubricant formulation is also to be employed for cooling, it can also comprise large amounts of water, in this case it being possible for the oil content in the lubricant formulation also to be significantly below the above-mentioned concentration ranges.
   In this fifth variant of the process according to the invention, the n-nonyl ester is preferably added to the lubricant formulation in the function of a solvent or in the function of a surfactant, it being preferable in this case for the n-nonyl ester to be employed in an amount of from 0.001 to 40 wt. %, particularly preferably from 0.1 to 30 wt. %, still more preferably from 1 to 20 wt. % and most preferably from 2 to 10 wt. %, in each case based on the total weight of the organic composition.
   The additives which, according to this fifth variant of the process according to the invention, can be provided in process step ic) can be, in particular, surface-active, oil-improving or oil-protecting additives. The surface-active additives include detergents, dispersing agents, high pressure or wear protection additives, corrosion and rust protection additives and additives which modify the coefficient of friction. The oil-improving additives modify the properties of the oil with respect to viscosity, pour point and its behavior towards elastomers, for example of seals. The oil-protecting additives have an antiageing effect on the oil, deactivate metal particles and prevent foaming of the oil. Very finely ground solids, such as, for example, Teflon (PTFE), ceramic oxides or molybdenum disulphide compounds, can also be added as additives. If the lubricant composition is also to be employed as a coolant, it can additionally comprise water in amounts of up to 95 wt. %, particularly preferably in amounts of up to 90 wt. %, in such a case the lubricant composition preferably being in the form of an emulsion.
6. According to a sixth variant of the process according to the invention, the functional component is a coloring agent and the organic composition is a lacquer or a paint. In the context of the present invention, a “paint” here is understood as meaning a non-glossy, open-pored coating with a high dyestuff and pigment content but only a low binder content, while a “lacquer” is understood as meaning a composition for coating surfaces of wood, metal, plastic or mineral material, which has a higher binder content compared with a paint.
   The coloring agent can be an inorganic or an organic coloring agent, it being possible for these coloring agents to be water-soluble or water-insoluble. Possible coloring agents are, in particular, inorganic or organic, preferably pulverulent pigments. Pigments differ from dyestuffs in that they are insoluble in their use media. Suitable inorganic pigments and their mode of preparation can be found in G. Buxbaum; “Industrial Inorganic Pigments”, 1st ed., p. 85-107; VCH Verlagsgesellschaft mbH, Weinheim, 1993, G. Buxbaum; “Industrial Inorganic Pigments”, 1st ed., p. 114-117; VCH Verlagsgesellschaft mbH, Weinheim, 1993 and G. Buxbaum; “Industrial Inorganic Pigments”, 1st ed., p. 124-131; VCH Verlagsgesellschaft mbH, Weinheim, 1993. The disclosure of these publication with respect to the inorganic pigments is introduced herewith as reference and forms part of the disclosure of the present invention. Suitable organic pigments and their mode of preparation can be found, in particular, in W. Herbst and K. Hunger; “Industrielle organische Pigmente”; 2nd ed.; P. 4-11; VCH Verlagsgesellschaft mbH, Weinheim, 1995, W. Herbst and K. Hunger; “Industrielle organische Pigmente”; 2nd ed.; P. 462; VCH Verlagsgesellschaft mbH, Weinheim, 1995, W. Herbst and K. Hunger; “Industrielle organische Pigmente”; 2nd ed.; P. 482-485; VCH Verlagsgesellschaft mbH, Weinheim, 1995, W. Herbst and K. Hunger; “Industrielle organische Pigmente”; 2nd ed.; P. 503; VCH Verlagsgesellschaft mbH, Weinheim, 1995 and W. Herbst and K. Hunger; “Industrielle organische Pigmente”; 2nd ed.; P. 567-569; VCH Verlagsgesellschaft mbH, Weinheim, 1995. Suitable classes of organic coloring agents (orientated on the parent substance of the colored structural unit) which may be mentioned are nitroso, nitro, monoazo, diazo, triazo, stilbene, diphenylmethane, triarylmethane, xanthene, acridine, quinoline, thiazole, indamine, azine, oxazine, thiazine, lactone and phthalocyanine coloring agents.
   The lacquer or paint can comprise these coloring agents in amounts in a range of from 0.001 to 40 wt. %, particularly preferably in amounts in a range of from 0.01 to 30 wt. %, still more preferably in amounts in a range of from 0.01 to 30 wt. % and most preferably in amounts in a range of from 0.1 to 10 wt. %, in each case based on the total weight of the organic composition.
   In this sixth variant of the process according to the invention, the n-nonyl ester is preferably added to the lacquer or paint in the function of a solvent or in the function of a surfactant, it being preferable in this case for the n-nonyl ester to be employed in an amount of from 0.001 to 40 wt. %, particularly preferably from 0.1 to 30 wt. %, still more preferably from 1 to 20 wt. % and most preferably from 2 to 10 wt. %, in each case based on the total weight of the organic composition.
   The additives which, according to this sixth variant of the process according to the invention, can be provided in process step ic) can be, in particular, binders, such as, for example, plant oils, balsam resin from coniferous trees, casein from milk, alkyd resin, polyurethane resin or epoxy resin, solvents, such as, for example, water, ethanol, citrus peel oil, white spirit or glycol ethers, thixotropy agents, antioxidants, viscosity regulators, skin and foam preventers, flow agents, UV absorbers, extenders, preservatives or binders. The amount of additives to be employed can vary within wide ranges. This applies in particular to the binders and solvents, depending on whether the composition is a lacquer or a paint.
7. According to a seventh variant of the process according to the invention, the functional component is a hair or skin care substance and the organic composition is a cosmetic formulation.
   Examples of hair and/or skin care substances which may be mentioned in particular are 18-β-glycyrrhetic acid from liquorice root extract (Gylcyrrhiza glabra), preferably in a purity of >99% pure substance in the extract, aescin in horse chestnut (Aesculus hippocastanum), allantoin, Aloe vera (containing chiefly sugars, anthraquinones and minerals, such as zinc), amino acids, such as, for example, alanine, arginine, serine, lysine, ammonium glycyrrhizate from liquorice root extract, preferably in a purity of almost 100% pure substance in the extract, apigenin from camomile extract (Matricaria recutita), Arnica, in particular Arnica montana or Arnica chamissonis, asiaticosides and madecassosides in Centella asiatica extract, avenanthramides from oat extract (Avena sativa), avocadol, azulene from camomile extract (Matricaria recutita), biotin (vitamin H), bisabolol from camomile extract (Matricaria recutita), brown algae extract (Ascophyllum nodosum), chlorogenic acid in the aqueous extract of Japanese honeysuckle (Lonicera japonica), coenzyme Q10, creatine, dexpanthenol, disodium glycyrrhizate from liquorice root extract, preferably in a purity of almost 100% pure substance in the extract, extract from red algae (Asparagopsis armata), flavonoids from birch extract (Betula alba), flavonoids, vitexin in the extract of the passionflower (Passiflora incarnata), flavonoids, vitexin in lime extract (Tilia platyphyllos), ginkgo flavone glycosides and terpene lactones in ginkgo extract (Ginkgo biloba), ginsenosides in ginseng extract (Panax ginseng), glycogen, grapefruit extract, witch hazel extract from virgin witch hazel (Hamamelis virgiana), honey, isoflavone glycosides in clover extract (Trifolium pratense), St.-John's-wort extract from St.-John's-wort (Hypericum perforatum), jojoba oil, lecithin, maize oil (Zea mays), evening primrose oil, niacinamide, oenothein B in the extract from willow herb (Epilobium angustifolium), oleuropein in olive extract (Olea europea), phytocohesin (sodium beta-sitosterol sulphate), plankton extract (Tetraselmis suecica, Spirulina and others), polyphenols, catechols from the extract of grape seeds (Vitis vinifera), polyphenols, catechols from green tea (Camellia sinensis), marigold extract (Calendula officinalis), rosemary acid in Melissa extract (Melissa officinalis), sea buckthorn oil, β-glucans from oats (Avena sativa), stearylglycyrrhetic acid (stearyl ester of 18-β-glycyrrhetic acid), sterols, sitosterol in stinging nettle extract (Urtica dioca), sweet almond oil (Prunus dulcis), vitamin C and its esters, vitamin E and its esters, wheat germ oil, zinc gluconate/magnesium aspartate/copper gluconate, and zinc sulphate or zinc oxide and proteins or protein derivatives, such as, for example, protein hydrolysates (for example collagen, keratin, silk protein or wheat protein hydrolysates).
   The cosmetic formulations can comprise these hair and/or skin care substances in amounts in a range of from 0.001 to 40 wt. %, particularly preferably in amounts in a range of from 0.01 to 30 wt. %, still more preferably in amounts in a range of from 0.01 to 30 [sic] wt. % and most preferably in amounts in a range of from 0.1 to 10 wt. %, in each case based on the total weight of the organic composition.
   In this seventh variant of the process according to the invention, the n-nonyl ester is preferably added to the cosmetic formulation in the function of a solvent or in the function of a surfactant, it being preferable in this case for the n-nonyl ester to be employed in an amount of from 0.001 to 40 wt. %, particularly preferably from 0.1 to 30 wt. %, still more preferably from 1 to 20 wt. % and most preferably from 2 to 10 wt. %, in each case based on the total weight of the organic composition.
   Suitable additives which, according to this seventh variant of the process according to the invention, can be provided in process step ic) are to be found, for example, in Schrader, K., “Grundlagen and Rezepturen der Kosmetika”, 2nd edition, 1989, pages 728-737, Domsch, A., “Die kosmetischen Priiparate”, Verlag für chemische Industrie (H. Ziolkowsky, ed.), 4th edition, volume 2 pages 212-230, 1992 or Johnson, D. H., “Hair and Hair Care”, New York, 1997, pages 65-104. The additives can be used in the conventional amounts known to the person skilled in the art, in particular in amounts of from 0.1 to 10.0 wt. %, based on the total weight of the organic composition.

A contribution towards achieving the abovementioned objects is also made by a process for the production of a shaped article, comprising the process steps:
I) provision of a thermoplastic composition obtainable by the process described above according to the first variant;
II) heating of the thermoplastic composition to the glass transition temperature of the thermoplastic polymer or to a temperature above the glass transition temperature of the thermoplastic polymer;
III) production of a shaped article from the heated thermoplastic composition prepared in process step II).

In step I) of the process according to the invention for the production of a shaped article, a thermoplastic composition according to the invention is first provided, this provision preferably being carried out by a process according to the first variant of the process according to the invention.

In process step II), the thermoplastic composition is then heated to the glass transition temperature of the thermoplastic polymer or to a temperature above the glass transition temperature of the thermoplastic polymer. In this connection, it is in turn preferable for the heating of the thermoplastic composition to be carried out to a temperature in a range of from 5 degrees below the glass transition temperature (T_{g}) to 100° C. above the glass transition temperature of the thermoplastic polymer employed, particularly preferably to a temperature in a range of from 1 degree below the glass transition temperature (T_{g}) to 50° C. above the glass transition temperature of the thermoplastic polymer employed and most preferably to a temperature in a range of from 1 degree above the glass transition temperature (T_{g}) to 20° C. above the glass transition temperature of the thermoplastic polymer employed, here also the upper limit of the temperature range being essentially limited by the decomposition temperature of the thermoplastic polymer employed.

In principle, process steps I) and II) can be carried out simultaneously or in succession. It is appropriate to carry out process steps I) and II) simultaneously, for example, if the thermoplastic composition is prepared by means of a melt mixing process. Where appropriate, it may be advantageous here to convert the composition prepared by the melt mixing process directly into a shaped article. It is appropriate to carry out process steps I) and II) successively, for example, if the thermoplastic composition is prepared by means of a dry mixing process or if the thermoplastic composition is indeed prepared by means of a melt mixing process, but is not subjected to the formation of a shaped article directly after the preparation, but rather is first cooled according to process step v).

In process step III) of the process according to the invention for the production of a shaped article, a shaped article is produced from the heated thermoplastic composition prepared in process step II). Possible processes for the production of a shaped article are, in particular, injection molding, extrusion molding, compression molding, layer molding, laminating molding, blow molding, vacuum molding and transfer molding, injection molding being particularly preferred.

An embodiment of the process according to the invention for the production of a thermoplastic shaped article furthermore comprises at least one further process step IV) in which at least a part region of the shaped article obtained in process III) serves as a shaped article blank and is reduced in its mass cross-section compared with that of the shaped article obtained in process step III). The mass cross-section is the cross-section of a region of the shaped article made solidly from the thermoplastic molding composition according to the invention. For example, in containers or vessels, the mass cross-section is the thickness of a wall of this container or vessel. In the case of shaped articles which are rather thread- or strand-like in construction, the mass cross-section is the thickness of these threads or strands. In the case of rather planar structures, such as sheets, layers, webs, films or foils, the mass cross-section is the thickness of these planar structures. For the reduction in the mass cross-section, in principle all the methods known to the person skilled in the art and suitable for this are possible. These include, for example, stretching in one or two directions, drawing in one or two directions, centrifugation or blowing, each of which are preferably carried out at elevated temperatures at which the thermoplastic composition according to the invention is so soft or even liquid that stretching, drawing, centrifugation or blowing can be carried out. The part region in which the reduction in cross-section is effected preferably makes up at least 50% and particularly preferably at least 80% of the shaped article obtained in step III). Stretching or drawing are generally carried out if a fiber is to be obtained from the shaped article obtained in step III). For the production of films, on the one hand drawing or stretching in one or more dimensions can be carried out. Thus, the web running out of an extruder can be drawn on to a roll at a higher speed compared with the exit speed from the extruder. On the other hand, if a container or vessel is to be obtained, apart from stretching, drawing and centrifugation, blowing is chiefly carried out in step IV). In this, the reduction in mass cross-section is effected by applying a gas pressure. The gas pressure is generally chosen such that the thermoplastic composition, which is usually heated at least to the glass transition temperature, of the shaped article obtained in step III) can be extended. The extending is as a rule limited by using a mould having the final shape of the shaped article. In addition to containers, such as freezer boxes, dishes and packaging for foodstuffs, such as fruit, vegetables or meat, and medicaments as tablets, capsules, suppositories or powders, vessels for liquids can also be produced in this way. As well as for liquids of the cosmetic or pharmaceutical industry, these vessels for liquids can also be used in the foodstuffs industry, preferably in the drinks industry, as reusable vessels, such as PET or PLA bottles. It is furthermore possible for two or more of process steps I) to IV) to be supplemented by further process steps and/or to at least overlap in time. This applies in particular to process steps III) and IV).

In addition to bottles, other shaped articles can furthermore also be produced according to the invention. These include disposable and reusable vessels, such as plates, dishes, pots or beakers, and cutlery, such as knives, forks or spoons. The biodegradable thermoplastic compositions according to the invention are particularly suitable for these uses.

A contribution towards achieving the abovementioned objects is furthermore made by a process for the production of a packed product, comprising as process steps:
a) provision of a product and a shaped article, in particular a film, the shaped article being obtainable by the process described above;
b) at least partial surrounding of the product with the shaped article.

The product provided in process step a) is preferably a pharmaceutical, a body care composition, an agricultural auxiliary substance, an adhesive, a building material, a dyestuff or a foodstuff.

The at least partial surrounding of the product can be carried out, for example, by the process described in DE-A-103 56 769.

A contribution towards achieving the abovementioned objects is also made by a process for coating substances which can be consumed by living beings, comprising as process steps:
A) provision of a substance which can be consumed by living beings, for example a foodstuff or a medicament, and of an n-nonyl ester, which is obtainable by reaction of an n-nonyl alcohol component with a further component which is capable of reacting with the n-nonyl alcohol component to form an n-nonyl ester;
B) at least partial surrounding of the substance which can be consumed by living beings with the n-nonyl ester.

The provision of the n-nonyl ester is preferably carried out according to process step ia) of the process described above for the preparation of an organic composition.

The at least partial surrounding of the substance which can be consumed by living beings with the n-nonyl ester can be carried out, for example, in a manner such that the substance which can be consumed and the n-nonyl ester are mixed with one another in suitable mixing devices, possible mixing devices being, in particular, the Patterson-Kelley mixer, DRAIS turbulence mixer, Lodige mixer, Ruberg mixer, screw mixers, plate mixers and fluidized bed mixers as well as continuously operating vertical mixers, in which the polymer structure is mixed by means of rotating blades in rapid frequency (Schugi mixer). Should the n-nonyl ester not be liquid under the mixing conditions, this component is to be heated to a temperature above the melting temperature of the n-nonyl ester before or during the mixing with the substance which can be consumed by living beings. In addition to the use of the mixing devices described above, the at least partial surrounding of the substance which can be consumed by living beings with the n-nonyl ester can also be carried out, for example, by initially introducing the substance which can be consumed by living beings into a fluidized bed mixer and spraying the n-nonyl ester in liquid form on to the substance which can be consumed by living beings.

A contribution towards achieving the abovementioned objects is also made by the use of at least one n-nonyl ester, which is obtainable by reaction of an n-nonyl alcohol component with a further component which is capable of reacting with the n-nonyl alcohol component to form an n-nonyl ester, as an additive in a composition comprising as a functional component

α) a thermoplastic polymer, wherein the composition is a thermoplastic composition;

β) an enzyme, wherein the composition is a detergent;

γ) a setting agent of an adhesive, wherein the composition is an adhesive;

δ) a paraffin, wherein the composition is a defoamer;

ε) an oil, wherein the composition is a lubricant formulation;

ζ) a coloring agent, wherein the composition is a lacquer or a paint; or
η) a hair or skin care substance, wherein the composition is a cosmetic formulation;

wherein the n-nonyl ester has preferably been obtained by the process described above for the preparation of an n-nonyl ester comprising process steps ia1), ia2), ia3) and optionally ia4).

A contribution towards achieving the abovementioned objects is also made by the use of the n-nonyl ester described above, which is obtainable by reaction of an n-nonyl alcohol component with a further component which is capable of reacting with the n-nonyl alcohol component to form an n-nonyl ester, as an additive in compositions employed during drilling of bore holes.

It is particularly preferable according to the invention for the n-nonyl ester described above to be used as an additive in drilling fluids or cleaning compositions for drilling equipment.

The invention therefore also relates to a process for cleaning the surfaces of bore holes, in particular the walls of bore holes, of conveyor pipes or casings or of walls of the casing, and for cleaning drilling equipment or drillings, wherein the surfaces are first brought into contact with a cleaning composition comprising the n-nonyl ester described above and the surfaces are then optionally rinsed off with water.

In this connection, it is preferable in particular for the cleaning composition to be employed in the form of an aqueous solution, an aqueous dispersion or an oil-in-water emulsion containing
(α1) 0.1 to 50 wt. %, particularly preferably 0.5 to 35 wt. %, further preferably 1.0 to 15 wt. % and most preferably 1.2 to 10 wt. % of the n-nonyl ester,
(α2) 0 to 50 wt. %, particularly preferably 0.5 to 35 wt. %, further preferably 1.0 to 15 wt. % and most preferably 1.2 to 10 wt. % of further additives which differ from the n-nonyl ester, and
(α3) 1 to 99.9 wt. %, particularly preferably 30 to 99 wt. %, further preferably 70 to 98 wt. % and most preferably 80 to 97.6 wt. % of water,

wherein the sum of components (α1) to (α3) is 100 wt. %.

In particular, the amount of component (α1) in the aqueous composition can vary and is adapted to the nature and the extent of the contamination.

Possible additives (α2) which differ from the n-nonyl ester are, in particular, weighting agents, fluid-loss additives, viscosity-regulating additives, wetting agents or salts. The general regulations for the composition of the particular treatment liquids apply here.

The co-use of organic polymer compounds of natural and/or synthetic origin may also prove to be advantageous. There are to be mentioned here in particular starch or chemically modified starches, cellulose derivatives, such as carboxymethylcellulose, guar gum, synthan gum or also purely synthetic water-soluble and/or water-dispersible polymer compounds, in particular of the type of high molecular weight polyacrylamide compounds with or without anionic or cationic modification.

Drilling equipment includes, in particular, drilling apparatuses such as, for example, the drilling rig, the drill string, in particular the drill rods and the drill bit, cleaning installations, installations for disposal of solids, in particular shaking screens or centrifuges, pumps, motors or gearing systems, or the drilling platform or parts thereof. For cleaning the drilling equipment, the cleaning composition comprising the n-nonyl ester is sprayed on or applied to the surfaces of the objects, or the objects to be cleaned are immersed in the aqueous compositions. The contamination thereby becomes detached from the surfaces. The surfaces are then brought into contact with water such that the compositions are removed together with the contamination, for example by spraying down the surface with a jet of water.

The cleaning composition comprising the n-nonyl ester can furthermore be used for cleaning drillings, the so-called “drill cuttings”. These are produced during boring and in the case of off-shore drillings must be deposited on the sea-bed surrounding the drilling platform, which can lead to a high introduction of mineral oil into the environment. In order largely to avoid ecological stress on the sea, the cuttings are cleaned beforehand and freed from residues of the drilling fluid. The cleaning composition comprising the n-nonyl ester can be used for all the cleaning operations known to the person skilled in the art which arise in the field of drilling into the earth, both in off-shore drillings and in drillings on land. These include, in particular, the removal of paraffin deposits from bore hole walls. Bore holes are conventionally cleaned by pumping a cleaning liquid through the bore hole under pressure and removing the deposits from the walls of the bore hole by means of the cleaning composition. The contamination is then transported out of the bore hole with the liquid.

According to a preferred embodiment of the process according to the invention described above, this comprises the process steps
(β1) drilling of a bore hole into the earth by means of a drill head driven by a drill rod,
(β2) introduction of a casing into the bore hole, and
(β3) introduction of cement into at least a part region of the intermediate space between the outside of the casing and the walls of the bore hole,

wherein before process step (β3) is carried out, the cleaning composition comprising the n-nonyl ester is passed through the intermediate space between the outside of the casing and the walls of the bore hole, preferably is circulated in this intermediate space. This circulation can take place, for example, by a procedure in which the cleaning composition is pumped downwards through the casing, preferably via the drill rod, exits at the lower end of the casing, preferably at the drill head or at the drill bit, and then rises upwards again through the intermediate space between the outside of the casing and the walls of the bore hole. If the cleaning composition is pumped continuously downwards through the casing, both the walls of the bore hole and the outside of the casing can be cleaned in this manner.

According to a preferred embodiment of the process according to the invention for cleaning the surfaces of drilling equipment, this comprises the process step of drilling a bore hole into the earth by means of a drill head driven by a drill rod, wherein the cleaning composition comprising the n-nonyl ester is led at least partly through the drill head, preferably is circulated at least partly through this, this passing through or this circulating taking place at least partly during the presence of the drill head in the bore hole.

Possible drilling equipment of which the surface can be cleaned with the cleaning composition is in turn, in particular, drilling apparatuses such as, for example, the drilling rig, the drill string, in particular the drill rods and the drill bit, cleaning installations, installations for disposal of solids, in particular shaking screens or centrifuges, pumps, motors or gearing systems, or the drilling platform or parts thereof.

A contribution towards achieving the abovementioned objects is also made by a process for the production of a bore hole, comprising the process steps
(β1) drilling of a bore hole into the earth by means of a drill head driven by a drill rod,
(β2) introduction of a casing into the bore hole,
(β3) introduction of cement into at least a part region of the intermediate space between the outside of the casing and the walls of the bore hole,
(β4) optionally introduction of a conveyor pipe into the casing,
(β5) optionally introduction of a sealing liquid into the intermediate space between the outside of the conveyor pipe and the inside of the casing,

wherein surfaces of the bore hole, the guide pipe, the drill rod or the drill head are brought into contact with the cleaning composition comprising the n-nonyl ester. In particular, this bringing into contact can be carried out according to the preferred embodiment described above for the process according to the invention, for cleaning the surfaces of bore holes or drilling equipment. It is accordingly preferable, before process step (β3) is carried out, for the cleaning composition comprising the n-nonyl ester to be passed through the intermediate space between the outside of the casing and the walls of the bore hole, preferably to be circulated through this intermediate space.

All the materials known to the person skilled in the art for this purpose can be employed as the sealing liquid which is introduced into the intermediate space between the outside of the conveyor pipe and the inside of the casing in process step (β5). Those sealing liquids which are described in U.S. Pat. No. 7,219,735 may be mentioned as an example at this point.

A further contribution towards achieving the abovementioned objects is also made by a process for the production of an oil or a gas which, in addition to the above-mentioned process steps (β1) to (β3) and optionally (β4) and (β5), also comprises the process steps
(β6) conveying of oil or gas through the bore hole, and
(β7) purifying or refining of the oil or gas conveyed,

wherein here also the surfaces of the bore hole, the conveyor pipe, the drill rod or the drill head are brought into contact with the cleaning composition comprising the n-nonyl ester. Here also, this bringing into contact can be carried out according to the preferred embodiment described above for the process according to the invention, for cleaning the surfaces of bore holes or drilling apparatuses.

The invention also relates to a process for the production of bore holes, in which a drilling fluid is pumped through a bore hole, wherein a composition comprising the n-nonyl ester described above is used as the drilling fluid.

According to a particular embodiment of this process, this composition is a water-in-oil emulsion.

In this connection, it is preferable in particular for the composition to contain
I) 28.9 to 99 wt. %, particularly preferably 60 to 90 wt. % and most preferably 70 to 80 wt. %, in each case based on the total weight of the composition, of a water-immiscible organic oil phase,
II) 1 to 48 wt. %, preferably, particularly preferably 5 to 40 wt. % and most preferably 10 to 30 wt. %, in each case based on the total weight of the composition, of water or aqueous phase,
III) 0.1 to 20 wt. %, particularly preferably 1 to 15 wt. % and most preferably 5 to 10 wt. %, in each case based on the total weight of the composition, of the n-nonyl ester described above, and
IV) 0 to 70 wt. %, particularly preferably 1 to 5 wt. % and most preferably 1.5 to 3 wt. %, in each case based on the total weight of the composition, of at least one further additive,

wherein the sum of components I) to IV) is 100 wt. %.

In connection with the water-in-oil emulsion described above, it is preferable for the organic oil phase I) to be chosen entirely or in part from the group of
a) paraffins having 5 to 22 C atoms and/or
b) paraffins having 5 to 22 C atoms and/or
c) internal olefins having 12 to 30 C atoms in the molecule and/or
d) carboxylic acid esters of the general formula R—COO—R′, in which R represents a linear or branched, saturated or unsaturated alkyl radical having 15 to 25 C atoms and R′ denotes a saturated, linear or branched alkyl radical having 3 to 22 C atoms, and/or
e) mineral oils, and/or
f) linear alpha-olefins (LAOS) having 12 to 30 C atoms, and/or
g) carbonates.

In this connection it is furthermore preferable for this water-in-oil emulsion to have a density of the liquid component in a range of from 1.2 to 3.0 g/cm³ and in particular in a range of from 1.5 to 3.0 g/cm³. The oil phase of the systems according to the invention comprises components a) to e) by themselves or components a), b), d) or e) together in a mixture with esters c) and optionally in a mixture with other suitable oil phases. Any desired mixtures of the oil phases a) to e) with one another are also possible.

Component a)

According to the invention, linear or branched paraffins having 5 to 22 C atoms are employed as component a). As is known, paraffins—more correctly called alkanes—are saturated hydrocarbons which, for the linear and branched representatives, follow the general empirical formula CₙH₂₊₁. The cyclic alkanes follow the general empirical formula CₙH₂ₙ. The linear and branched paraffins are particularly preferred, whereas cyclic paraffins are less preferred. The use of branched paraffins is preferred in particular. Those paraffins which are liquid at room temperature, that is to say those having 5 to 16 C atoms per molecule, are furthermore preferred. However, it may also be preferable to employ paraffins having 17 to 22 C atoms, which have a wax-like consistency. It is preferable, however, to employ mixtures of the various paraffins, it being particularly preferable for these mixtures still to be liquid at 21° C. Such mixtures can be formed e.g. from paraffins having 10 to 21 C atoms. Paraffins are particularly preferred oil phases—by themselves or as a mixture constituent with further oil phases—in drilling fluids—preferably those of the invert type, in which the crosslinked glycerol or oligoglycerol esters according to the invention are used as thickeners.

Component b)

Internal olefins (abbreviated to IO in the following) can be employed according to the invention as component b). In this context, IOs are likewise compounds which are known per se and can be prepared by all the processes know for this to the person skilled in the art. EP 0 787 706 A1 describes e.g. a process for the synthesis of IOs by isomerization of alpha-olefins on sulphonic or persulphonic acids. It is characteristic that the IO obtained in this way are linear and comprise at least one olefinic double bond which is not in the alpha-position of the alkyl chain. Those IO or IO mixtures which comprise IO having 12 to 30 C atoms in the molecule, preferably having 14 to 24 C atoms and in particular having up to 20 C atoms in the molecule are preferably used according to the invention.

Component c)

Esters of the general formula R—COO—R′, in which R represents a linear or branched, saturated or unsaturated alkyl radical having 15 to 25 C atoms and R′ denotes a saturated, linear or branched alkyl radical having 6 to 22 C atoms, are furthermore a constituent of the oil phases according to the invention. Such esters are also known chemical compounds. The main use thereof in drilling fluids is e.g. the subject matter of EP 0 374 672 A1 and EP 0 374 671 A1. The use of those esters of which the radical R represents a saturated or unsaturated alkyl radical having 15 to 25 C atoms and R′ represents a saturated alkyl radical having 3 to 10 C atoms is particularly preferred. The saturated compounds are preferred in particular in this context. In the context of the teaching according to the invention, it is preferable for the oil phase to comprise, in addition to the esters according to the above description, a maximum of 15 wt. % (based on the oil phase) of other esters with radicals R which represent alkyl radicals having more than 23 C atoms.

Component d)

Mineral oils are a collective term for liquid distillation products which are obtained from mineral raw materials (crude oil, brown and hard coal, wood or peat) and essentially comprise mixtures of saturated hydrocarbons. The mineral oils preferably contain only small amounts of aromatic hydrocarbons, preferably less than 3 wt. %. Mineral oils which are based on crude oil and are liquid at 21° C. are preferred. The mineral oils preferably have boiling points of from 180 to 300° C.

Component e)

Linear alpha-olefins (LAO for short) are unbranched hydrocarbons which are unsaturated in the 1-position (“alpha C atom”). They can be based on natural substances, but in particular are also widely obtained by synthesis. LAO based on natural substances are obtained as linear products with an even carbon number by dehydration of fatty alcohols based on natural substances. The LAO obtained by synthesis routes—prepared by oligomerization of ethylene—often contain even carbon numbers in the chain, but processes for the preparation of uneven-numbered alpha-olefins are nowadays also known. In the context of the definition according to the invention—because of their volatility—as a rule at least 10, preferably at least 12 to 14 C atoms occur in the molecule. The upper limit of the LAO which are flowable at room temperature is in the range of from C₁₈ to C₂₀. However, this upper limit is not limiting for the usability of this substance class in the context of the invention. The upper limit of suitable LAO compounds for use in the context of the teaching according to the invention is thus significantly above the abovementioned limit value of C₁₈ to C₂₀ and can reach, for example, C₃₀.

Component f)

In the context of the present application, carbonates are understood as meaning carbonic acid esters of fatty alcohols having 8 to 22 C atoms, preferably the diesters of carbonic acid. Such compounds and the use thereof as an oil phase for drilling fluids are described in DE 40 18 228 A1.

In addition to components a) to f), the oil phase I) can also contain other water-insoluble constituents, as long as these are ecologically acceptable. Further particularly suitable mixture constituents of the oil phase I) according to the invention are therefore specifically:
(i) esters of C₁₋₅-monocarboxylic acids and 1- and/or polyfunctional alcohols, wherein radicals of 1-hydric alcohols have at least 6, preferably at least 8 C atoms and the polyhydric alcohols preferably have 2 to 6 C atoms in the molecule,
(ii) mixtures of secondary esters chosen from the group of propyl carboxylate, butyl carboxylate, pentyl carboxylate, hexyl carboxylate, heptyl carboxylate, octyl carboxylate, nonyl carboxylate, decyl carboxylate, undecyl carboxylate, dodecyl carboxylate, tridecyl carboxylate, tetradecyl carboxylate, pentadecyl carboxylate, hexadecyl carboxylate, heptadecyl carboxylate, octadecyl carboxylate, nonadecyl carboxylate, eicosyl carboxylate, uneicosyl carboxylate, doeicosyl carboxylate and isomers thereof, wherein the secondary esters in each case have a carboxylate radical having 1 to 5 C atoms, water-insoluble ethers of monohydric alcohols having 6 to 24 C atoms,
(iii) water-insoluble alcohols having 8 to 36 C atoms
(iv) poly-alpha-olefins (PAO)
(v) mixtures of components (i) to (iv).

The oil phase I) of the composition in the form of a water-in-oil emulsion employed as a drilling fluid preferably has a pour point below 0° C., preferably below −5° C. (measured in accordance with DIN ISO 3016: 1982-10). The Brookfield viscosity of the oil phase at 0° C. is at most 50 mPas. The compositions employed as a drilling fluid have, if they are formed as an oil-based drilling fluid of the W/O type, a plastic viscosity (PV) in the range of from 10 to 70 mPas and a yield point (YP) of from 5 to 60 lb/100 ft², in each case determined at 50° C. The kinematic viscosity of the oil phase, measured by the Ubbelohde method at 20° C., should preferably be at most 12 mm²/sec. The aqueous phase of the compositions according to the invention preferably has a pH in the range of from 7.5 to 12, preferably from 7.5 to 11 and in particular from 8 to 10.

The composition employed as a drilling fluid preferably comprises as the aqueous phase according to component II) aqueous salt solutions, preferably saturated salt solutions, it being possible for all the alkali metal or alkaline earth metal halides known to the person skilled in the art to be employed as salts. Examples of suitable salts which may be mentioned are, in particular, KCl, NaCl, LiCl, KBr, NaBr, LiBr, CaCl₂, and MgCl₂, among these CaCl₂, NaCl and KCl or mixtures of these salts being particularly preferred.

Possible further additives which the composition employed as a drilling fluid can comprise according to component IV) are, in particular, additives chosen from the group consisting of surfactants as an admixing component for the crosslinked glycerol or oligoglycerol esters, weighting agents, fluid-loss additives, pH modifiers, further viscosity-modifying additives, wetting agents, salts, biocides, agents for inhibition of undesirable exchange of water between drilled formations—e.g. water-swellable clays and/or salt beds—and the e.g. water-based fluid, wetting agents for better absorption of the emulsified oil phase on solid surfaces, e.g. to improve the lubricating action, but also to improve the oleophilic closure of exposed rock formations or rock faces, corrosion inhibitors, alkali reserves and emulsifiers.

The general regulations for the composition of the particular treatment liquids for which data are given by way of example in the following with the aid of corresponding drilling muds apply here. The additives can be water-soluble, oil-soluble and/or water- or oil-dispersible.

Surfactants which can be used are anionic, nonionic, zwitterionic or cationic surfactants. However, the nonionic and the anionic surfactants are preferred. Typical examples of anionic surfactants are soaps, alkylbenzenesulphonates, alkanesulphonates, olefinsulphonates, alkyl ether sulphonates, glycerol ether sulphonates, methyl ester sulphonates, sulpho fatty acids, alkyl sulphates, fatty alcohol ether sulphates, glycerol ether sulphates, fatty acid ether sulphates, hydroxy-mixed ether sulphates, monoglyceride (ether) sulphates, fatty acid amide (ether) sulphates, mono- and dialkyl sulphosuccinates, mono- and dialkyl sulphosuccinamates, sulphotriglycerides, amide soaps, ether carboxylic acids and salts thereof. The latter are particularly preferred surfactant components in the context of the present technical teaching. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, optionally partially oxidized alk(en)yl oligoglycosides or glucuronic acid derivatives, fatty acid N-alkylglucamides, polyol fatty acid esters, sugar esters, sorbitan esters, polysorbates and amine oxides. If the nonionic surfactants contain polyglycol ether chains, these can have a conventional, but preferably a narrowed distribution of homologues. The surfactants are an optional constituent in the additives. They are preferably employed in amounts of from 0.01 to 2 wt. %, in particular from 0.1 to 1.5 wt. % and preferably from 0.2 to 0.5 wt. %, in each case based on the total water-in-oil emulsion.

Possible emulsifiers are, preferably, nonionic emulsifiers which are assigned in particular to one of the following substance classes: (oligo)alkoxylates—in particular lower alkoxylates, where corresponding ethoxylates and/or propoxylates are of particular importance here—of base molecules of natural and/or synthetic origin which contain lipophilic radicals and are capable of alkoxylation. Alkoxylates of the type mentioned are known as such—i.e. with a terminal free hydroxyl group on the alkoxylate radical—to be nonionic emulsifiers, but the corresponding compounds can also be closed by end groups, for example by esterification and/or etherification. A further important class of nonionic emulsifiers for the purposes of the invention are partial esters and/or partial ethers of polyfunctional alcohols having in particular 2 to 6 C atoms and 2 to 6 OH groups and/or oligomers thereof with acids and/or alcohols containing lipophilic radicals. In this context, compounds of this type which are also suitable in particular are those which additionally contain, bonded into their molecular structure, (oligo)alkoxy radicals and in this context in particular corresponding oligoethoxy radicals. The polyfunctional alcohols having 2 to 6 OH groups in the base molecule or the oligomers derived therefrom can be, in particular, diols and/or triols or oligomerization products thereof, where glycol and glycerol or their oligomers can be of particular importance. Known nonionic emulsifiers of the ethylene oxide/propylene oxide/butylene oxide block polymer type are also to be assigned to the field of partial ethers of polyfunctional alcohols. A further example of corresponding emulsifier components are alkyl (poly)glycosides of long-chain alcohols and the already mentioned fatty alcohols of natural and/or synthetic origin or alkylolamides, amine oxides and lecithins. The co-use of the now commercially available alkyl (poly)glycoside compounds (APG compounds) as emulsifier components in the context according to the invention may be of particular interest, inter alia, because this is an emulsifier class of particularly pronounced ecological acceptability. Without claim to completeness, from the substance classes of suitable emulsifier components listed here, the following representatives are additionally mentioned: (oligo)alkoxylates of fatty alcohols, fatty acids, fatty amines, fatty amides, fatty acid and/or fatty alcohol esters and/or ethers, alkanolamides, alkylphenols and/or reaction products thereof with formaldehyde and further reaction products of carrier molecules containing lipophilic radicals with lower alkoxides. As stated, the particular reaction products can also be closed by end groups at least in part. Examples of partial esters and/or partial ethers of polyfunctional alcohols are, in particular, the corresponding partial esters with fatty acids, for example of the type of glycerol mono- and/or diesters, glycol monoesters, corresponding partial ester of oligomerized polyfunctional alcohols, sorbitan partial esters and the like, and corresponding compounds with ether groupings.

The co-use of organic polymer compounds of natural and/or synthetic origin as further additives can be of considerable importance in this connection. There are to be mentioned here in particular starch or chemically modified starches, cellulose derivatives, such as carboxymethylcellulose, guar gum, synthan gum or also purely synthetic water-soluble and/or water-dispersible polymer compounds, in particular of the type of high molecular weight polyacrylamide compounds with or without anionic or cationic modification. Thinners for regulating the viscosity: The so-called thinners can be organic or inorganic in nature, examples of organic thinners are tannins and/or quebracho extract. Further example of these are lignite and lignite derivatives, in particular lignosulphonates.

The preferred agent against fluid loss (fluid-loss additive) is, in particular, organophilic lignite, while preferred pH modifiers can be found, for example, in EP 0 382 701 A1. The invention described in EP 0 382 701 A1 is based on the knowledge that in ester-based drilling fluids of the water-in-oil type, additives which ensure that the rheological properties of the drilling fluid also do not change when increasing amounts of free carboxylic acids are released due to partial hydrolysis of the esters should be added. These free carboxylic acids should as far as possible be converted into compounds which have stabilizing and emulsifying properties. For this purpose, EP 0 382 701 A1 proposes the addition of alkaline amines of high oleophilicity and the lowest possible water-solubility which are capable of reacting with the free acids to form salts. Typical examples of such amine compounds are primary, secondary and/or tertiary amines which are predominantly water-insoluble and which furthermore can be at least partly alkoxylated and/or substituted by hydroxyl groups. Further examples include aminoamides and/or heterocyclic compounds which contain nitrogen as a ring atom. Suitable compounds are, for example, basic amines which have at least one long-chain hydrocarbon radical having 8 to 36 carbon atoms, preferably having 10 to 24 carbon atoms, it also being possible for these hydrocarbon radicals to be mono- or polyunsaturated.

The amounts in which the further additives of the composition employed as a drilling fluid which are described above are added in the case of water-in-oil emulsion conventionally correspond to those amounts in which these compounds are added to the drilling fluids on a water-in-oil basis which are known from the prior art.

In compositions which have little weighting, component IV) is preferably a weighting agent, such as, for example, BaSO₄, component IV) preferably being employed in an amount of up to 20 wt. % in the case of a composition which has little weighting. In more highly weighted compositions, component IV) is preferably employed in an amount of from 20 to 50 wt. %, while in highly weighted compositions 50 to 70 wt. % of component IV) can be employed.

It is furthermore preferable according to the invention for the composition, if it is present as a water-in-oil emulsion, to be a nanoemulsion or a microemulsion which preferably comprises drops of water or drops of an aqueous phase having a drop size of less than 1,000 μm, preferably having a drop size in a range of from 5 nm to 1,000 μm, particularly preferably having a drop size in a range of from 10 nm to 850 μm, still more preferably having a drop size in a range of from 20 nm to 700 μm, still more preferably having a drop size in a range of from 50 nm to 500 μm. According to the invention, the terms “microemulsion” and “nanoemulsion” characterize emulsions which comprise drops in the micrometer or nanometer range, it being possible for there to be a certain overlapping of these two ranges and therefore also of these two terms. According to some of the technical literature, and also of the prior art relating to drilling fluids, microemulsions are preferably understood as meaning those emulsions which form spontaneously on combination of the emulsion components, whereas the formation of nanoemulsions conventionally requires supplying of energy, for example in the form of homogenization, in particular in the form of high pressure homogenization.

In the case of a water-in-oil emulsion as a composition employed as a drilling fluid, this can be prepared by any process known to the person skilled in the art for the preparation of such a water-in-oil emulsion. It is thus conceivable, in particular, first to prepare the base emulsion from the organic oil phase as the continuous phase and the drops of water emulsified therein, and only then to add the above n-nonyl ester and optionally the further additives. However, it is also conceivable first to add the n-nonyl ester described above to the organic oil phase and then to form the emulsion from this oil phase and the water or the aqueous solution.

According to another particular embodiment of the composition employed as a drilling fluid, this is an aqueous solution or an oil-in-water emulsion.

In this connection, it is preferable in particular for the composition to contain
I) 0 to 48 wt. %, particularly preferably 0.1 to 20 wt. % and most preferably 1 to 10 wt. %, in each case based on the total weight of the composition, of a water-immiscible organic oil phase,
II) 29.9 to 99.9 wt. %, particularly preferably 60 to 99 wt. % and most preferably 70 to 95 wt. %, in each case based on the total weight of the composition, of water or aqueous phase,
III) 0.1 to 20 wt. %, particularly preferably 1 to 15 wt. % and most preferably 5 to 10 wt. %, in each case based on the total weight of the composition, of the n-nonyl ester described above,
IV) 0 to 70 wt. %, particularly preferably 1 to 5 wt. % and most preferably 1.5 to 3 wt. %, in each case based on the total weight of the composition, of at least one further additive,

wherein the sum of components I) to IV) is 100 wt. %.

Those organic oil phases, aqueous phases and further additives which have already been mentioned above in connection with the water-in-oil emulsion are preferred as the organic oil phase, aqueous phase and further additives.

In the case also of an oil-in-water emulsion as a composition employed as a drilling fluid, this can be prepared by any process known to the person skilled in the art for the preparation of such an oil-in-water emulsion. It is thus conceivable, in particular, first to prepare the base emulsion from water or the aqueous solution as the continuous phase and the drops of the oil phase emulsified therein, and only then to add the n-nonyl ester described above and optionally the further additives. However, it is also conceivable first to add the n-nonyl ester described above to the organic oil phase and then to form the emulsion from this oil phase and the water or the aqueous solution.

According to a preferred embodiment of this process for the production of bore holes in which a drilling fluid is pumped through a bore hole, this comprises the process steps:
(α1) provision of the composition according to the invention, in particular the composition according to the invention in the form of a water-in-oil emulsion, an aqueous solution or an oil-in-water emulsion;
(α2) drilling of a hole into the earth;
(α3) introduction, preferably circulation, of the composition provided in process step (α1) at least partly into or in the bore hole;

wherein the introduction, preferably the circulation, preferably takes place at least partly during the drilling in process step (α2).

The composition according to the invention consequently acts as a drilling fluid during drilling of holes into the earth, preferably when drilling for crude oil or natural gas.

A contribution towards achieving the abovementioned objects is consequently also made by a process for the production of an oil or gas, comprising the process steps:
(α1) provision of the composition employed as a drilling fluid, in particular the composition employed as a drilling fluid in the form of a water-in-oil emulsion, an aqueous solution or an oil-in-water emulsion;
(α2) drilling of a hole into the earth;
(α3) introduction, preferably circulation, of the composition provided in process step (α1) at least partly into or in the bore hole, here also the introduction or circulation preferably taking place at least partly during the drilling in process step (α2);
(α4) conveying of oil or gas from the earth through the hole drilled in process step (α2);
(α5) optionally purifying or refining of the oil or gas conveyed in process step (α3).

A contribution towards achieving the abovementioned objects is also made by a cleaning composition and a drilling fluid, preferably a drilling fluid in the form of the water-in-oil emulsion described above or the oil-in-water emulsion described above.

The invention is now explained in more detail with the aid of non-limiting examples.

### EXAMPLE 1

### Preparation of Oleic Acid N-Nonyl Ester

31.6 g of pelargonic acid (0.2 mol, Emery® 1203) and 150 ml of methanol were initially introduced into a glass flask and 3 g of conc. sulphuric acid were added. The mixture was boiled under reflux for 4 hours. Thereafter, 3.5 g of anhydrous sodium carbonate were added and the excess alcohol was distilled off. The pelargonic acid methyl ester was distilled off in vacuo (p approx. 16 mbar) at 95-100° C.

6 wt. % of copper chromite catalyst was added to 29.2 g of the pelargonic acid methyl ester obtained in this way and the mixture was stirred in an autoclave at 230° C. under a hydrogen pressure of 250 bar for 4 hours. Thereafter, the catalyst was filtered off and the filtrate was distilled in vacuo. The boiling point was about 113° C. under 26 mbar and the yield was 79%.

The batch described above was repeated several times for the preparation of n-nonanol.

346.9 g of the n-nonanol obtained in this way and 421 g of technical grade oleic acid (EDENOR Ti05) were initially introduced into a flask with a distillation bridge and 0.38 g of tin(II) oxalate (Fluka) was added. The reaction mixture was heated from 150° C. to 220° C. in the course of 3 hours. Thereafter, a vacuum was slowly applied and, after a further 2 hours at 220° C. (acid number of the reaction mixture=1.0), the excess n-nonanol was distilled off in vacuo. The mixture was cooled to 90° C. and filtered.

### EXAMPLE 2

### Preparation of Stearic Acid N-Nonyl Ester

347 g of n-nonanol (prepared analogously to Example 1) and 409 g of technical grade stearic acid (EDENOR ST1) were initially introduced into a flask with a distillation bridge and 0.38 g of tin(II) oxalate (Fluka) was added. The reaction mixture was heated from 150° C. to 220° C. in the course of 3 hours. Thereafter, a vacuum was slowly applied and, after a further 3 hours at 220° C. (acid number of the reaction mixture=0.5), the excess n-nonanol was distilled off in vacuo. The mixture was cooled to 90° C. and filtered.

### EXAMPLE 3

### Preparation of a Thermoplastic Composition

6 kg of polyethylene terephthalate (PET SP04 from Catalana de Polimers) were introduced into a 15 kg Henschel mixer. The mixing wall temperature was 40° C. 0.5 wt. % of the n-nonyl ester prepared in Example 2 was furthermore added as a mould release agent. The material was then granulated on a granulator (ZSK 24Mcc) with a stuffing screw.

For production of shaped articles from the thermoplastic composition, a fully hydraulic injection molding machine with a hydraulic closing unit of the Battenfeld HM800/210 type was employed. The maximum closing force is 800 KN, the screw diameter is 25 mm. A mould with a conically tapering, rectangular core was used at the test mould. For determination of the demolding force, a load cell with a maximum measuring range of 2 kN was attached to the ejector rod. The molding composition was predried at about 225° C. for about 4 hours. Significantly improved demolding was observed with the thermoplastic composition according to the invention compared with a molding composition without a mould release agent.

### EXAMPLE 4

### Preparation of a Detergent

0.2 wt. % of zinc ricinoleate (Tego Sorb Conc 50 from Goldschmidt), 1 wt. % of sodium citrate, 0.1 wt. % of the n-nonyl ester obtained in Example 1, as a defoamer, 1 wt. % of boric acid, 7.5 wt. % of glycerol, 1 wt. % of ethanol, 4 wt. % of C₁₂-C₁₆-alkyl glycoside, 8 wt. % of soap, 8 wt. % of C₁₂-C₁₄-fatty alcohol+1.3 EO sulphate sodium salt, 1 wt. % of Acusol 120 (15%; methacrylic acid (stearyl alcohol-20 EO) ester/acrylic acid copolymer from Rohm & Haas), 0.5 wt. % of Dequest 2066, amylase, protease, and water were mixed to give a detergent.

### EXAMPLE 5

### Preparation of an Adhesive

A high molecular weight diisocyanate was prepared from a polypropylene glycol of Mn=880 and diphenylmethane-diisocyanate in accordance with the teaching of DE-A-199 57 351, and the monomeric MDI was then removed this until a residual monomer content of 0.1% resulted. A hot-melt adhesive was prepared from 100 parts of a polyol mixture for a standard polyurethane hot-melt adhesive (QR 6202, Henkel) having an average OH number of 32.5 and 76.5 parts of the abovementioned high molecular weight diisocyanate. 5 wt. % of the n-nonyl ester prepared in Example 2 was additionally added.

### EXAMPLE 6

### Preparation of a Defoamer

4.0 wt. % of paraffin having a solidification point in accordance with DIN ISO 2207 of 45° C., a liquid content at 40° C. of about 66 wt. % and a liquid content at 60° C. of about 96 wt. %, 1.2 wt. % of bisamide, 3 wt. % of sodium carbonate, 58.7 wt. % of sodium sulphate, 21.4 wt. % of sodium silicate, 2.1 wt. % of cellulose ether, 4.8 wt. % of the n-nonyl ester obtained in Example 1 and water are mixed to form an aqueous slurry which was spray dried with superheated steam by the process of the European patent specification EP 625 922.

### EXAMPLE 7

### Preparation of a Defoamer Based on N-Nonyl Ester

1.2 wt. % of bisamide, 3 wt. % of sodium carbonate, 58.7 wt. % of sodium sulphate, 21.4 wt. % of sodium silicate, 2.1 wt. % of cellulose ether, 8.8 wt. % of the n-nonyl ester obtained in Example 1 and water are mixed to form an aqueous slurry which was spray dried with superheated steam by the process of the European patent specification EP-A-0 625 922.

### EXAMPLE 8

### Preparation of a Textile Auxiliary Substance

5 g of the polymer emulsion prepared according to Example 1b of DE-A-39 39 549 were added to 995 g of a textile lubricant comprising 78.5 wt. % of i-butyl stearate, 5 wt. % of oleyl/cetyl alcohol 5 mol EO, 2.2 wt. % of coconut fatty acid monoethanolamide 4 mol EO, 0.8 wt. % of oleic acid, 6 wt. % of the n-nonyl ester obtained in Example 2, 6 wt. % of secondary fatty alcohol 7 mol EO (Tergitol 1587, manufacturer: Union Carbide) and 1.5 wt. % of water at 20° C., while stirring (maximum stirring speed of an overhead stirrer with a propeller stirrer). After 30 seconds, the polymer emulsion had become uniformly distributed and a clear solution was formed. Thereafter, the stirring speed was reduced as far as possible and the textile lubricant was heated to 60° C. to accelerate dissolving of the polymer particles.

### EXAMPLE 9

### Preparation of a Lacquer

736 g of demineralized water, 4 g of a 70 wt. % solution of stearic acid isodecyl ester in C₁₂H₂₆ (isomer mixture), 10 g of sodium nitrobenzenesulphonate, 5 g of the tetrasodium salt of ethylenediaminetetraacetic acid, 100 g of urea, 25 g of sodium bicarbonate, 100 g of D-I.1 and 20 g of Fluorescent Brightener C.I. 230 were initially introduced into a mixing vessel. 5 g of the n-nonyl ester obtained in Example 1 were added as a defoamer and the mixture was stirred with a high-speed stirrer at 2,000 rpm for 60 seconds.

### EXAMPLE 10

### Preparation of a Cosmetic Formulation

An 0/W emulsion was prepared, the oil phase of which had the following composition:
5.0 g of the compounds characterized by the formula (I) in EP-A-1 485 061, in which R′ represents methyl and R in each case represents a butyloctanoyl radical (C₁₂),
5.0 g of the emulsifier dioctyl ether (Cetiol OE, Cognis),
0.6 g of the emulsifier cetyl stearyl alcohol+20 EO (Eumulgin B2, Cognis),
0.1 g of creatine.

5 wt. % of the n-nonyl ester obtained in Example 1 was added to the composition obtained in this way.

### EXAMPLE 11

### Preparation of a Drilling Fluid

A conventional lime-treated fluid was prepared from 7.6 g of prehydrated bentonite, 1.15 g of ferrochrome lignosulphonate, 2.3 g of slaked lime, 0.38 g of starch and 0.76 g of NaOH. 5 wt. % of the n-nonyl ester obtained in Example 1 was added to this lime-treated fluid.

1. A process for the preparation of an organic composition which comprises a functional component chosen from thermoplastic polymer, an enzyme, a setting agent, a paraffin, an oil, a coloring agent and a hair or skin care substance, comprising as process steps of:
i) providing
ia) an n-nonyl ester as an additive, which is obtainable by reaction of an n-nonyl alcohol component with a further component which is capable of reacting with the n-nonyl alcohol component to form an n-nonyl ester,
ib) the functional component, and optionally
ic) at least one further additive; and
ii) mixing of the n-nonyl ester, the functional component and optionally the at least one further additive.

2. The process according to claim 1, wherein the further component which is capable of reacting with the n-nonyl alcohol component to form an n-nonyl ester is a mono-, di-, tri-, tetra- or polycarboxylic acid having more than four carboxyl groups, a derivative of such a carboxylic acid or a mixture of such a carboxylic acid and a derivative of such a carboxylic acid.

3. The process according to claim 2, wherein the carboxylic acid is chosen from the group consisting of adipic acid, trimellitic acid, terephthalic acid and azelaic acid or a mixture of at least two of these.

4. The process according to claim 1 wherein the at least one further additive is employed in an amount in a range of from about 0.001 to about 40 wt. %, based on the composition.

5. The process according to claim 1 wherein the n-nonyl alcohol component is obtained from pelargonic acid to the extent of at least about 80 wt. %, based on the n-nonyl alcohol component.

6. The process according to claim 5, wherein the pelargonic acid is obtained from oleic acid.

7. The process according to claim 1 wherein the n-nonyl alcohol component contains less than about 10 wt. %, based on the n-nonyl alcohol component, of C₈- and C₁₀-alcohols.

8. The process according to claim 1 wherein the n-nonyl ester is an alkoxylated n-nonyl ester having 2 to 30 ether recurring units.

9. The process according to claim 1 wherein the functional component is a thermoplastic polymer and a thermoplastic composition is obtained as the composition.

10. A process for the production of a shaped article, comprising the process steps:
I) providing a thermoplastic composition obtainable by the process according to claim 9;
II) heating the thermoplastic composition to the glass transition temperature of the thermoplastic polymer or to a temperature above the glass transition temperature of the thermoplastic polymer;
III) producing a shaped article from the heated thermoplastic composition prepared in process step II).

11. The process according to claim 10, wherein in a further process step IV) at least a part region of the shaped article obtained in process step III) is reduced in its mass cross-section compared with process step III).

12. The process according to claim 11 wherein the shaped article is chosen from a group consisting of a container, a film, a fiber or at least two thereof.

13. A process for the production of a packed product, comprising as process steps:
a) providing a product and a shaped article obtainable by a process according to claim 10; and
b) at least partial surrounding of the product with the shaped article.

14. A process for coating substances which can be consumed by living beings, comprising as process steps:
A) providing a substance which can be consumed by living beings and of an n-nonyl ester, which is obtainable by reaction of an n-nonyl alcohol component with a further component which is capable of reacting with the n-nonyl alcohol component to form an n-nonyl ester; and
B) at least partial surrounding of the substance which can be consumed by living beings with the n-nonyl ester.

15. Use of at least one n-nonyl ester, which is obtainable by reaction of an n-nonyl alcohol component with a further component which is capable of reacting with the n-nonyl alcohol component to form an n-nonyl ester, as an additive in a composition containing as a functional component
α) a thermoplastic polymer, wherein the composition is a thermoplastic composition;
β) an enzyme, wherein the composition is a detergent;
γ) a setting agent of an adhesive, wherein the composition is an adhesive;
δ) a paraffin, wherein the composition is a defoamer;
ε) an oil, wherein the composition is a lubricant formulation;
ζ) a colouring agent, wherein the composition is a lacquer or a paint; or
η) a hair care or skin care substance, wherein the composition is a cosmetic formulation.

16-20. (canceled)

21. Use of an n-nonyl ester, which is obtainable by reaction of an n-nonyl alcohol component with a further component which is capable of reacting with the n-nonyl alcohol component to form an n-nonyl ester, as an additive in compositions employed in the drilling of bore holes.

22. The use according to claim 21, wherein the n-nonyl ester is used as an additive in drilling fluids or cleaning compositions for drilling equipment.

23-28. (canceled)

29. A process for cleaning the surfaces of bore holes, drilling equipment or drill cuttings, wherein the surfaces are first brought into contact with a cleaning composition comprising an n-nonyl ester as defined in claim 21 and the surfaces are then optionally rinsed off with water.

30. A process according to claim 29, comprising the process steps:
(β1) drilling of a bore hole into the earth by means of a drill head driven by a drill rod,
(β2) introducing a casing into the bore hole, and
(β3) introducing cement into at least a part region of the intermediate space between the outside of the casing and the walls of the bore hole.
wherein before process step (β3) is carried out, the cleaning composition comprising an n-nonyl ester as defined in claim 21 is passed through the intermediate space between the outside of the casing and the walls of the bore hole, preferably is circulated in this intermediate space.

31. The process according to claim 29, comprising the process steps
(β1) drilling of a bore hole into the earth by means of a drill head driven by a drill rod,
wherein the cleaning composition comprising an n-nonyl ester as defined in claim 21 is passed at least partly through the drill head, this passing through at least partly taking place during the presence of the drill head in the bore hole.

32. A process for the production of a bore hole, comprising the process steps
(β1) drilling of a bore hole into the earth by means of a drill head driven by a drill rod,
(β2) introducing a casing into the bore hole,
(β3) introducing cement into at least a part region of the intermediate space between the outside of the casing and the walls of the bore hole.
(β4) optionally introducing a conveyor pipe into the casing,
(β5) optionally introducing a sealing liquid into the intermediate space between the outside of the conveyor pipe and the inside of the casing,
wherein surfaces of the bore hole, the guide pipe, the drill rod or the drill head are brought into contact with a cleaning composition comprising an n-nonyl ester as defined in claim 21.

33. A process for the production of an oil or a gas, comprising the process steps:
(β1) drilling of a bore hole into the earth by means of a drill head driven by a drill rod,
(β2) introducing a casing into the bore hole,
(β3) introducing cement into at least a part region of the intermediate space between the outside of the casing and the walls of the bore hole.
(β4) optionally introducing a conveyor pipe into the casing,
(β5) optionally introducing a sealing liquid into the intermediate space between the outside of the conveyor pipe and the inside of the casing,
(β6) conveying of oil or gas through the bore hole, and
(β7) purifying or refining of the oil or gas conveyed,
wherein surfaces of the bore hole, the guide pipe, the drill rod or the drill head are brought into contact with a cleaning composition comprising an n-nonyl ester as defined in claim 21.

34. A process for the production of bore holes, in which a drilling fluid is pumped through a bore hole, wherein a composition comprising an n-nonyl ester as defined in claim 21 is used as the drilling fluid.

35. The process according to claim 34, wherein the composition is a water-in-oil emulsion.

36. The process according to claim 35, wherein the composition contains:
I) about 28.9 to about 99 wt. %, based on the total weight of the composition, of a water-immiscible organic oil phase,
II) about 1 to about 48 wt. %, based on the total weight of the composition, of water or aqueous phase,
III) about 0.1 to about 20 wt. %, based on the total weight of the composition, of the n-nonyl ester defined in claims 21 and 23 to 28,
IV) 0 to about 70 wt. %, based on the total weight of the composition, of at least one further additive,
wherein the sum of components I) to IV) is 100 wt. %.

37. The process according to claim 35 wherein the water-in-oil emulsion is a nanoemulsion or a microemulsion which comprises drops of water or drops of an aqueous phase having a drop size in a range of from about 5 nm to about 1,000 μm.

38. The process according to claim 34, wherein the composition is an aqueous solution or an oil-in-water emulsion.

39. The process according to claim 38, wherein the composition contains
I) 0 to about 48 wt. %, based on the total weight of the composition, of a water-immiscible organic oil phase,
II) about 29.9 to about 99.9 wt. %, based on the total weight of the composition, of water or aqueous phase,
III) about 0.1 to about 20 wt. %, based on the total weight of the composition, of the n-nonyl ester defined in claims 21 and 23 to 28,
IV) 0 to about 70 wt. %, based on the total weight of the composition, of at least one further additive,
wherein the sum of components I) to IV) is 100 wt. %.

40. The process according to claim 36 wherein the at least one further additive is an additive chosen from the group consisting of thickening agents, clays, agents against loss of liquid, pH modifiers, viscosity modifiers, agents for control of filtration, emulsifiers, salts, wetting agents, weighting agents and dispersing agents.

41. The process according to claim 34 comprising the process steps:
(α1) provision of a composition as defined in claim 35;
(α2) drilling of a hole into the earth;
(α3) introducing, preferably circulation, of the composition provided in process step (α1) at least partly into and/or in the bore hole.

42. The process according to claim 41, wherein the introduction, preferably the circulation, takes place at least partly during the drilling in process step (α2).

43. A process for the production of an oil or a gas, comprising the process steps:
(α1) provision of a composition as defined in claim 35;
(α2) drilling of a hole into the earth;
(α3) introducing, preferably circulation, of the composition provided in process step (α1) at least partly into and/or in the bore hole;
(α4) conveying of oil or gas from the earth through the hole drilled in process step (α2);
(α5) optionally purifying or refining of the oil or gas conveyed in process step (α3).
